(19) United States
(12) Patent Application Publication
(10) Pub. No.: US 2003/0224021 A1
(43) Pub. Date: Dec. 4, 2003

(54) Methods of using epitope peptides of human pathogens

(75) Inventor: Bianca M. Conti-Fine, Minneapolis, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Regents of the University of Minnesota

(21) Appl. No.: 10/356,765

(22) Filed: Jan. 30, 2003

Related U.S. Application Data
(63) Continuation of application No. 09/199,748, filed on Nov. 25, 1998, now abandoned.

(51) Int. Cl. A61K 39/05

(52) U.S. Cl. 424/245.1

(57) ABSTRACT

Isolated and purified T cell epitope peptides and variants thereof, useful to immunize a mammal, e.g., a human, against an infectious pathogen are provided. Also provided are methods to identify and use the peptides.

### CROSS-REFERENCE TO RELATED APPLICATION

This application is a continuation of U.S. patent application Serial No. 09/199,748, filed Nov. 25, 1998, currently pending, which is incorporated by reference herein.

### BACKGROUND OF THE INVENTION

Immunization describes the process of administering antigen to a live host for the purpose of inducing an immune response. Vaccines were developed as a prophylactic measure to prevent disease caused by infectious agents and, provided their use caused only low levels of morbidity and especially mortality, this was initially the sole criterion for their effectiveness. When methods for the quantitative estimation of antibody were developed, the practice of estimating seroconversion, that is, the levels of antibody before and after immunization, came into general use. In many situations (e.g., influenza infection), vaccines may have preexisting antibody titers and in the absence of a natural challenge, the success of vaccination is judged by the extent of increase in the level of specific antibody. With experience of a particular disease, this could be used to predict a vaccine's efficacy. In some cases, however, there was not always a correlation between seroconversion and protection, and there is now the recognition that cell-mediated immune responses are important in protection from disease and might not parallel antibody responses. Another criterion for efficacy is whether the immune response in vaccinees after challenge is characteristic of a secondary immune response.

Diphtheria is caused by Corynebacterium diphtheriae (Collier et al., Bacteriol. Rev., 39, 54 (1975)). The organism secretes a catalytic protein, diphtheria toxin (DTX), which is a potent exotoxin that is transported in the blood of an infected organism to remote tissues, causing hemorrhagic and necrotic damage to those tissues in susceptible organisms. DTX is a single chain of 535 amino acids (Greenfield et al., PNAS USA, 80, 6853 (1983)), which, upon mild trypsinization and reduction in vitro breaks into fragments A (21 kDa) and B (37 kDa) (Collier et al., J. Biol. Chem., 246, 1496 (1971); Moskaug et al., J. Biol. Chem., 264, 15709 (1989)).

In the cytoplasm, the A fragment catalyzes ADP-ribosylation of a translationally modified histidine residue (diphthamide) on elongation factor-2, leading to the arrest of protein synthesis (Collier et al., In: ADP Ribosylation Reactions: Biology and Medicine, Academic Press, Inc., NY, p. 573 (1982)). While DTX is quite immunogenic, only anti-DTX IgG can inactivate the biologic activity of DTX. Inactivation depends on the antibody having a greater affinity for the toxin than the toxin has for its substrate. Thus, only high affinity hyperimmune IgG can achieve anti-toxin activity. The production of high affinity IgG requires, in the vast majority of cases, and specifically in the case of DTX, interaction of B cells with antigen-specific T helper (CD4+) cells.

Because diphtheria mortality is due to the effects of DTX, the key component of anti-diphtheria vaccines is diphtheria toxoid (DTD), a partially denatured, non-toxic form of DTX. Mass vaccination against diphtheria is carried out in virtually every country and entails several injections of vaccine to establish a good level of immunity, followed by periodic boosts at 10 or more years apart, during adult life.

While the existing anti-diphtheria vaccine preparations contain substantial amounts of bacterial impurities, they are highly effective in inducing high affinity antibody titers. However, these vaccines cause substantial undesirable side effects in a high percentage of immunized individuals. Severe side effects include convulsions (1/1750 doses), collapse (1/1750 doses), and acute encephalopathy (1/110,000 doses) which can result in permanent neurological damage. Less serious side effects include fever, pain, swelling and inconsolable crying in 10-15% of immunized children.

10-14% of adults also experience side effects, again ranging from mild to severe, primarily the result of sensitization to Corynebacterium proteins or toxin, or other corynebacteria. The majority of severe reactions in adults correlates with the administration of the standard dose of the vaccine, i.e., 12 units (Scheibel et al., Acta Pathol. et Microbiol. Scand., 27, 69 (1950)). Such severe reactions can be reduced when a smaller dose, e.g., 1 unit; is used, however, a protective antibody response with the lower-dose vaccine is only obtained when multiple immunizations are employed. Such a vaccination protocol is frequently unsuccessful due to low compliance of healthy subjects. This low compliance and the decrease over time of protective antibody response after immunization has led to a resurgence in cases of diphtheria in adults.

For unimmunized adults, passive immunotherapy with diphtheria anti-toxin is the only specific and effective treatment. However, commercial preparations of anti-toxin are derived from immunized non-human mammals, thus, providing a risk of inducing sensitization or anaphylactic reactions when these preparations are used.

Hence, there is a need for a method to identify T cell epitope peptides useful to immunize mammals, e.g., humans, against infectious agents.

### SUMMARY OF THE INVENTION

Studies using synthetic peptide sequences of the muscle nicotinic acetylcholine receptor (AChR), the auto Ag in myasthenia gravis, and tetanus toxin (TTX), have identified epitopes recognized by human T-helper (Th or CD4+) cells (Protti et al., Immunol. Today, 14, 363 (1993); Demoiz et al., J. Immunol., 142, 394 (1989); Reece et al., J. Immunol., 151, 6175 (1993)). Moreover, studies with AChR and TTX demonstrated that some sequence regions in these antigens (Ags) comprise epitope(s) recognized by CD4+ cells in many or all of the subjects tested, irrespective of their HLA class II haplotype (Protti et al., Immunol. Today, 14, 363 (1993); Panina-Bordingnon et al., Eur. J. Immunol., 19, 2237 (1989); Diethelm-Okita et al., J. Inf. Dis., 175, 382 (1997)). A sequence which is recognized by human CD4+ cells irrespective of their HLA class II haplotype is termed a universal epitope sequence. As described hereinbelow, universal T cell epitope peptides of DTX were identified (see Example 1). Further support for the existence of universal T cell epitopes for DTX and TTX is also described below in Example 2.

Moreover, T cell clones specific for universal DTX epitope sequences were found to be promiscuous in their recognition of universal T cell epitope sequences. Further, as described in co-pending U.S. application Ser. No. 08/991,143, which is incorporated by reference herein, the majority of humans sensitized to factor VIII have CD4+ cells that recognize certain universal epitopes of factor VIII.

CD4+ T cells control antibody synthesis. In mammals, limited sets of epitopes for each antigen dominate the CD4+ T cell response, referred to as immunodominant T cell epitope sequences (hereinafter “immunodominant epitope sequences” or “immunodominant region sequences”). Moreover, as mentioned above, in humans CD4+ cells recognize universal epitope sequences. As T cell epitopes may comprise as few as 7 amino acid residues, a peptide having at least about 7 amino acid residues that correspond to an amino acid sequence present in a particular antigen and which residues include a T cell epitope, or a portion thereof, may be useful to immunize a mammal to an infectious agent that expresses the cognate antigen. Preferably, the antigen is a polypeptide encoded by the nucleic acid (genome) of the infectious agent, or is otherwise associated with the infectious agent. Preferably, the antigen is present on the surface or exterior of the infectious agent so that the antigen is recognized by the immune system. As at least one universal T cell epitope may be present for every antigen present on or associated with, and/or specific for, an infectious agent, immunodominant and/or universal epitope peptides comprising at least one universal T cell epitope may be administered so as to regulate a mammal's T cell and antibody response. Preferably, the antigen is that of a virus, bacterium, parasite or fungus, and more preferably, the antigen is that of a virus, bacterium or fungus. Preferably, the antigen is not an antigen of Plasmodium falciparum, e.g., the circumsporozoite protein thereof, or an exotoxin, e.g., DTX or TTX. These universal epitopes can be identified as sequences that are easily processed (e.g., degraded by proteases) from the native antigen, sequences that bind to class II molecules of different isotypes, e.g., DR, DP, and DQ, sequences that bind to different class II alleles, and/or sequences that induce the proliferation of CD4+ T cells and/or secretion of one or more cytokines by CD4+ T cells.

Therefore, the invention provides an isolated and purified peptide comprising an amino acid sequence substantially similar or identical to a portion of the amino acid sequence of an antigen from an infectious agent. Although it is preferred that the peptide is between about 7 and about 40 amino acid residues in length, it is also envisioned that immunogenic fragments of the peptide are within the scope of the present invention. Preferably, the antigen to which at least a portion of the amino acid sequence of the peptide corresponds is expressed on the surface of the infectious agent. Preferred antigens are those specific for viruses, bacteria, fungi, and the like. Hence, preferred peptides of the invention include peptides that are substantially similar or identical to a portion of the amino acid sequence of hemagglutinin (HA) of influenza, the G or F protein of Respiratory Syncytial Virus (RSV), sAg of Hepatitis B virus, herpes glycoprotein D, surface glycoprotein of rabies virus, the glycoprotein of retroviruses and lentiviruses such as HIV, and antigens of Vibrio cholerae and BCG. Other preferred peptides of the invention include peptides from antigens of Vaccinia, Varicella zoster virus, Polio virus, Cytomegalovirus, Hepatitis A virus, Measles virus, Adenovirus, Influenza virus (e.g., A and B), Yellow fever virus, Mumps virus, Dengue virus, Hepatitis B virus, Japanese B encephalitis virus, Rabies virus, Rotavirus, Herpes simplex viruses 1 and 2, Herpesvirus varicellae, and Parainfluenza virus, Mycobacterium leprae, Vibrio cholerae, Salmonella typhi, Bordetella pertussis, Streptococcus pneumoniae (pneumococcus), Hemophilus influenzae (type B), Clostridium tentani, Corynebacterium diphtheriae, Coccidioides immitis, Neisseria gonorrhoeae, Streptococcus group B, Plasmodium spp., Escherichia coli, Shigella spp., Streptococcus group A, and Neisseria meningitidis. Preferred epitope peptides are peptides of Pneumococcus, Rotavirus, group A Streptococcus, hepatitis C, poliovirus, Clostridium tentani, Corynebacterium diphtheriae, Mycobacterium tuberculosis, hantavirus, Ebola virus and other viruses causing hemorrhagic fever, Pertussis, Rubella, hepatitis A, and hepatitis B.

As described below, six synthetic DTX-specific peptides stimulated the proliferation of anti-diphtheria toxoid (DTD) CD4+ T cell lines or anti-DTD PBMC from many, or all, individuals, irrespective of the HLA-haplotype of the individual, as determined by proliferation assays. These peptides comprise the following amino acid sequences: (1) Pro-Val-Phe-Ala-Gly-Ala-Asn-Tyr-Ala-Ala-Trp-Ala-Val-Asn-Val-Ala-Gln-Val-Ile (SEQ ID NO: 2); (2) Val-His-His-Asn-Thr-Glu-Glu-Ile-Val-Ala-Gln-Ser-Ile-Ala-Leu-Ser-Ser-Leu-Met-Val (SEQ ID NO: 3); (3) Gln-Ser-Ile-Ala-Leu-Ser-Ser-Leu-Met-Val-Ala-Gln-Ala-Ile-Pro-leu-Val-Gly-Glu-Leu (SEQ ID NO: 4); (4) Val-Asp-Ile-Gly-Phe-Ala-Ala-Tyr-Asn-Phe-Val-Glu-Ser-Ile-Ile-Asn-Leu-Phe-Gln-Val-Val (SEQ ID NO: 5); (5) Gln-Gly-Glu-Ser-Gly-His-Asp-Ile-Lys-Ile-Thr-Ala-Glu-Asn-Thr-Pro-Leu-Pro-Ile-Ala (SEQ ID NO: 6); and (6) Gly-Val-Leu-Leu-Pro-Thr-Ile-Pro-Gly-Lys-Leu-Asp-Val-Asn-Lys-Ser-Lys-Thr-His-Ile (SEQ ID NO: 7).

These peptides are depicted conventionally, from the amino terminus (left end) to the carboxyl terminus (right end), and formally represent amino acid residues 271-290 (1); 321-340 (2); 331-350 (3); 351-370 (4); 411-430 (5); and 431-450 (6) of the diphtheria toxin secreted by Corynebacterium diphtheriae (SEQ ID NO: 1, Greenfield et al., PNAS USA, 80, 6853 (1993). These peptides can be prepared in large quantities and in high purity by chemical syntheses and thus are much less expensive and more readily obtained than a pure DTX-derived antigen.

Most CD4+ epitopes are within fragment B of DTX, as described below, while fragment A, which bears the toxic catalytic domain and is the active part of DTX immunoconjugates, is poorly recognized by CD4+ cells. Therefore, better DTX immunotoxins are hormonotoxins that contain fragment A only, thus minimizing undesirable CD4+ responses and optimizing the long-term efficacy of the conjugate.

Also provided is a method to identify an immunogenic epitope. The method comprises exposing cultured mammalian, e.g., human or rodent, immune cells, e.g., hematopoietic cells, peripheral blood mononuclear cells, spleen cells or lymph node cells, to at least one isolated and purified peptide, wherein the amino acid sequence of the peptide is substantially similar or identical to a portion of the amino acid sequence of an antigen that is expressed on the surface or exterior of the agent. Then it is determined whether or not the cultured immune cells, such as the cultured peripheral blood mononuclear cells, spleen cells or lymph node cells, proliferate relative to control peripheral blood mononuclear cells, spleen cells or lymph node cells which were not exposed to the peptide or any other antigenic stimulus. The cultured test spleen cells or lymph node cells are preferably obtained from a H2-class II knockout, human HLA class II transgenic mouse. The peripheral blood mononuclear cells are preferably obtained from the native organism or HuPBL-SCID mice.

Alternatively, or in addition to determining the proliferation of the cultured immune cells, the production of at least one cytokine from the immune cells, e.g., cultured peripheral blood mononuclear cells, spleen cells or lymph node cells, exposed to the peptide is compared to the production of at least one cytokine from control immune cells, e.g., peripheral blood mononuclear cells, spleen cells or lymph node cells, which were not exposed to the peptide or any other antigenic stimulus.

For example, as described hereinbelow, cultured peripheral blood mononuclear cells are exposed to at least one isolated and purified peptide, wherein the amino acid sequence of the peptide is substantially similar, homologous or identical to a portion of the amino acid sequence of diphtheria toxin. Then it is determined whether or not the cultured peripheral blood mononuclear cells proliferate relative to control peripheral blood mononuclear cells which were not exposed to the peptide or any other antigenic stimulus. Preferably, the peptide is synthesized in vitro, and also preferably the peptide comprises at least about 7 amino acid residues. It is preferred that the cultured peripheral blood mononuclear cells were previously stimulated in vitro, e.g., with diphtheria toxoid, diphtheria toxin, or diphtheria toxin-specific peptides. This stimulation can result in CD4+ T cell lines or cells enriched in CD4+ cells, specific for diphtheria toxin epitopes. It is also preferred that the cells are depleted of CD8+ cells. If the production of at least one cytokine is detected or determined, preferably the cytokine is gamma interferon, IL-2, IL-3, IL-4, IL-5 or IL-10.

Further provided is a method to identify an immunodominant region sequence in a peptide. The method comprises exposing each of at least a first and a second culture of mammalian, such as human or murine, peripheral blood mononuclear cells, spleen cells or lymph node cells to at least one isolated and purified peptide, wherein the MHC class II haplotype of the peripheral blood mononuclear cells, spleen cells or lymph node cells in at least the first and second of the cultures is different, and wherein the amino acid sequence of the peptide is substantially similar or identical to a portion of the amino acid sequence of an antigen of an infectious agent. Preferably, the antigen is expressed on the surface of the agent. Then it is determined whether or not the peripheral blood mononuclear cells, spleen cells or lymph node cells in any of the exposed cultures proliferates relative to control peripheral blood mononuclear cells, spleen cells or lymph node cells which were not exposed to the peptide or any other antigenic stimulus. A preferred embodiment of the invention employs peripheral blood mononuclear cells, spleen cells or lymph node cells from mammals, e.g., humans or mice, which were previously stimulated in vitro, or that were previously exposed in vivo, to the infectious agent, an antigen of the infectious agent or to a peptide which has antigen-specific sequences.

Alternatively, it is determined whether or not the peripheral blood mononuclear cells, spleen cells or lymph node cells in any of the exposed cultures produce at least one cytokine relative to control peripheral blood mononuclear cells, spleen cells or lymph node cells which were not exposed to the peptide or any other antigenic stimulus.

For example, Example 1 (below) describes a method to identify an immunodominant region sequence in a diphtheria toxin peptide. The method comprises exposing each of at least a first and a second culture of peripheral blood mononuclear cells to at least one isolated and purified peptide, wherein the HLA haplotype of the peripheral blood mononuclear cells in at least the first and second of the cultures is different, and wherein the amino acid sequence of the peptide is substantially similar or identical to a portion of the amino acid sequence of diphtheria toxin. Then it is determined whether or not the peripheral blood mononuclear cells in any of the exposed cultures produce at least one cytokine relative to control peripheral blood mononuclear cells which were not exposed to the peptide or any other antigenic stimulus. A more preferred embodiment of the invention employs peripheral blood mononuclear cells previously stimulated in vitro with the antigen, e.g. diphtheria toxoid, diphtheria toxin, or diphtheria toxin-specific peptides, to yield CD4+ T cell lines which are specific for the antigen or epitopes of the antigen, or highly enriched in CD4+ cells specific for the antigen or epitopes of the antigen. Preferably, the peptide is synthesized in vitro, and also preferably, the peptide comprises at least about 7 amino acid residues. If the production of at least one cytokine is detected or determined, preferably the cytokine is gamma interferon, IL-2, IL-3, IL-4, IL-5 or IL-10.

Yet another embodiment of the invention is a method to identify an immunodominant epitope sequence in a mammal. The method comprises contacting at plurality of samples with a panel of peptides. Each sample comprises T cells and antigen presenting cells obtained from an individual mammal. The panel of peptides together correspond to the entire sequence of a particular antigen. Preferably, the peptides comprise overlapping sequences, i.e., each peptide comprises a sequence which overlaps with a portion of the sequence of at least one other peptide, such as the two adjacent peptides. Each sample is contacted with one of the peptides. Then it is determined whether the T cells from the mammal proliferate in response to one of the peptides relative to a sample contacted with an unrelated peptide that does not comprise an immunodominant epitope sequence and/or a sample which is not contacted with a peptide. Alternatively, or in addition to determining the proliferation of T cells, the secretion of at least one cytokine may also be determined.

Another embodiment of the invention is a method to identify a universal epitope sequence useful to immunize a mammal, e.g., a human. The method comprises contacting at least two samples with at least one preselected peptide. One sample comprises T cells, spleen cells or lymph node cells obtained from a first individual mammal. The second sample comprises T cells, spleen cells or lymph node cells from a second mammal, wherein the genotype of the second mammal differs at the loci of the major histocompatibility complex (MHC) from the genotype at the MHC loci of the first mammal, and wherein the mammals are of the same species. Then it is determined whether or not the T cells, spleen cells or lymph node cells from each mammal proliferate or secrete at least one cytokine relative to (negative) control T cells, spleen cells or lymph node cells which were not exposed to a peptide or any other antigenic stimulus, and/or relative to T cells, spleen cells or lymph node cells exposed to a (negative) control peptide, i.e., one not having a universal epitope sequence. A peptide having a universal epitope sequence will induce the proliferation of T cells, spleen cells or lymph node cells from samples from a majority of mammals of the same species, mammals which differ at the MHC loci. Alternatively, or in addition to determining the proliferation of T cells, the secretion of at least one cytokine may also be determined.

The invention also provides a vaccine comprising an immunogenic amount of at least one peptide containing a universal epitope sequence which is combined with a physiologically acceptable, non-toxic liquid vehicle, which amount is effective to immunize a susceptible mammal against or sensitize T cells to an infectious agent. The peptide comprises an amino acid sequence substantially similar or identical to a portion of the amino acid sequence of an antigen specific for the infectious agent. The peptide is combined with a physiologically acceptable, non-toxic liquid vehicle, optionally comprising conventional vaccine adjuvants, or optionally comprising the (inactivated) infectious agent or cognate antigen, e.g., a native, denatured or partially denatured protein that contains the epitope sequence. The amount of peptide administered, preferably in combination with an amount of the (inactivated) infectious agent or an antigen of the agent that is present on the surface of the infectious agent, is effective to immunize a susceptible mammal against, or sensitize T cells to, the infectious agent.

Further provided is an immunogenic composition comprising a peptide associated with, e.g., coupled to, a non- or poorly immunogenic molecule. The peptide comprises an amino acid sequence substantially similar or identical to a portion of the amino acid sequence of an antigen from an infectious agent. The antigen is preferably expressed on the surface of the agent. Preferably, the peptide consists essentially of an amino acid sequence region that is present on the surface of crystallized surface antigen of the agent. The peptide is between about 7 and about 40 amino acid residues in length. For example, the peptide consists essentially of an amino acid sequence substantially similar or identical to a portion of the diphtheria toxin amino acid sequence. The peptide is between about 7 and about 40 amino acid residues in length and a portion of the amino acid sequence in the peptide contains a contiguous sequence of amino acid residues that form at least one alpha helix or a beta sheet in vitro or in vivo.

Also provided is an immunotoxin consisting essentially of fragment A of diphtheria toxin linked to a binding protein that can specifically bind to a particular cell population, wherein the binding protein is an antibody molecule or a portion thereof with binding activity, and a hormonotoxin consisting essentially of fragment A of diphtheria toxin linked to a binding protein that can specifically bind to a particular cell population, wherein the binding protein is a hormone molecule or a portion thereof with binding activity.

The present invention thus provides a method to immunize a mammal comprising the administration of an epitope peptide comprising a universal and/or immunodominant epitope sequence derived from a particular antigen from an infectious agent that causes or is associated with an indication, pathology or disease in the mammal. The amount administered is effective to prevent or inhibit at least one symptom of the indication, pathology or disease. Preferably, for humans, the peptide comprises a universal, immunodominant epitope sequence and is effective to immunize the human. The epitope peptide does not include the entire sequence of the antigen from which it is derived.

Thus, the invention also provides an immunogen comprising at least one isolated and purified epitope peptide having a universal and/or immunodominant epitope sequence and a physiologically compatible carrier, the administration of which to a mammal results in an immune response specific for the infectious agent having an antigen which comprises at least a portion of the peptide. It is preferred that the peptide contains a contiguous sequence of at least about 7 amino acids having substantial similarity or identity with the amino acid sequence of the antigen, and that the peptide is no more than about 40 amino acid residues in length, i.e., it represents a fragment of said antigen.

### BRIEF DESCRIPTION OF THE FIGURES

FIG. 1. Position of synthetic DTX specific peptides relative to the sequence of DTX (SEQ ID NO: 1, Greenfield et al., PNAS USA, 80, 6853 (1993)). Synthetic peptides which comprise an IRS are indicated by a numerical code which includes two numbers. The first number refers to the position of the first residue in the peptide on the DTX sequence, and the second number refers to the last residue in the peptide on the DTX sequence. The uncharged DTX sequence regions identified by Sette et al. (J. Immunol., 151, 3163 (1993)) are indicated by blackened boxes.

FIG. 2. Response of CD4+ lines to PHA and DTD. CD4+ lines (Table 2) were isolated by subjecting PBMC from seven healthy individuals to repeated cycles of DTD stimulation. The lines were then challenged in proliferation assays with either 10 μg/ml of phytohemagglutinin (PHA) or 10 μg/ml of DTD. The bars represent the average ±SD of triplicate cultures. The basal rate of cell proliferation of the lines in the presence of antigen-resenting cells (APC) but in the absence of the antigen (“Basal”) is shown, but was not subtracted from the response to PHA or DTD. The stimulation scale, i.e., cpm, is different for different lines.

FIG. 3. Recognition of synthetic DTX sequences by CD4+ enriched PBMC and anti-DTD cell lines derived from the same subject. CD4+ enriched PBMC or an anti-DTD CD4+ cell line derived from subject #4 were challenged in a proliferation assay with individual DTX synthetic sequences (10 μg/ml), as indicated along the abscissa. Proliferation assays were also conducted in the presence of IL-2 (10%), PHA (10 μg/ml), or DTD (10 μg/ml). Basal rates of proliferation were assessed by culturing CD4+ enriched PBMC or CD4+ cell lines plus APC without any stimulus (“Basal”). These basal rates were not subtracted from the rates observed in the presence of antigen (Ag). Data are averages ±SD of triplicate cultures, and they are arranged in order of decreasing intensity of response. IRS peptides are indicated by arrows. In the top panel, the peptides that induced a significant (p<0.01) response of the CD4+ enriched PBMC are indicated with an asterisk(*).

FIG. 4. Synthetic peptides of DTX recognized by anti-DTD CD4+ cell lines. CD4+ cell lines from the seven subjects were challenged in proliferation assays with individual synthetic DTX peptides, as indicated along the abscissa. The bars represent average ±SD of triplicate cultures. The basal rate of cell proliferation, in the absence of the antigenic stimulus but in the presence of APC, is reported (“basal). The basal rate was not subtracted from the response to the peptides. Asterisks (*) above the bars represent significant (p<0.005) responses, as assessed by a two-tailed student's t test. Although each subject had an individual pattern of peptide recognition, six peptides, indicated by checkered boxes, were recognized by all subjects.

FIG. 5. HLA class II restriction of DTX IRSs in two subjects. Anti-DTD CD4+ lines from subject #3 (panel a) and subject #1 (panel b) were challenged in a proliferation assay with IL-2 (10%), a 20-residue synthetic sequence unrelated to DTX (10 μg/ml, “Control”), or individual IRS containing peptides, as indicated along the abscissa. The bars represent average ±SD of triplicate cultures. The proliferation assays were carried out in the absence of anti-class II monoclonal antibody (mAb, black bars), or in the presence of mAb against DR, DQ, or DP molecules, as indicated. Asterisks (*) represent a significant (p<0.05) decrease in the response to the peptide when the mAb was present, as compared to the response in the absence of the mAb.

FIG. 6. DTX synthetic sequences containing an IRS. The IRSs were aligned according to binding motifs identified for the DRB1 0101, 0401, 0402, and 0404 alleles. Residues in boxes conform to the motifs proposed by Hammer et al. (Cell, 74, 197 (1993)), Hammer et al. (J. Exp. Med., 176, 1007 (1995)), Hammer et al. (J. Exp. Med., 181, 1847 (1995)), Hammer et al. (PNAS USA, 91, 4456 (1994)), and Sette et al. (J. Immunol., 151, 3163 (1993)).

FIG. 7. Codons for various amino acids.

FIG. 8. Exemplary amino acid substitutions.

FIG. 9. Promiscuous recognition of universal T cell epitope sequences by T cell clones propagated by stimulation with an individual DTX epitope peptide. A) Response of PBMC from a healthy human subject to DTD and individual overlapping synthetic peptides spanning the DTX sequence. The top panel in A reports TCR Vβ usage of the PBMCs of the subject. B) Five T cell clones were derived from the subject. Three clones were obtained by stimulation with DTX universal epitope peptide 271-290. Two other clones were obtained by stimulation with DTX universal epitope peptide 411-430. E73 is a 20 residue peptide unrelated to DTX and which does not form a universal epitope.

### DETAILED DESCRIPTION OF THE INVENTION

Definitions:

As used herein, the term “immunogenic” with respect to an infectious agent, antigen or a peptide, means that the agent, antigen, or peptide can induce peripheral blood mononuclear cells (PBMC) or other lymphoid cells to proliferate when those cells are exposed to the agent, antigen or peptide, relative to cells not exposed to the agent.

As used herein, the terms “isolated and/or purified” refer to in vitro preparation, isolation and/or purification of a peptide or nucleic acid molecule of the invention, so that it is not associated with in vivo substances, or is substantially purified from in vitro substances.

“Immunodominant” T cell epitopes (also referred to as immunodominant region sequences “IRS” or immunodominant epitope sequences, and which include immunodominant CD4+ cell epitopes) refer to a sequence of a protein antigen, or the proteinaceous portion of an antigen, that is strongly recognized by the T cells, e.g., CD4+, cells of a mammal, as detected by methods well known to the art, including methods described herein. An “immunodominant” epitope sequence is an amino acid sequence containing the smallest number of contiguous amino acid residues which are strongly recognized by T cells. “Strongly” recognized means that the peptide elicits a statistically significant response as compared to the background response to a non-related peptide from an antigen, and that such response is at least two times higher than the average response obtained for at least about ⅓ of the peptides which elicit the lowest response from the peptides employed to identify the immunodominant epitopes.

An epitope peptide of the invention may comprise more than one immunodominant epitope sequence, and may comprise sequences which do not contain an immunodominant epitope sequence. Sequences which do not contribute to an immunodominant epitope sequence can be present at either or both the amino- or carboxyl-terminal end of the peptide. The non-immunodominant epitope sequences preferably are no more than about 10 to about 20 peptidyl residues in toto, and either do not affect the biological activity of the peptide or do not reduce the activity of the peptide by more than about 10 to about 20%.

As used herein, a “universal” epitope sequence is an epitope that is recognized by CD4+ cells from a majority, preferably at least about 66%, more preferably at least about 70%, and even more preferably at least about 75%, of individuals within a population of a particular mammalian species that is genetically divergent at the immune response loci, e.g., at the HLA loci in humans. Thus, within the scope of the invention, a universal epitope comprises an amino acid sequence containing the smallest number of contiguous amino acid residues which are recognized by CD4+ cells from a majority of mammals from the same species which are genetically different at their immune response loci. A peptide of the invention may comprise more than one universal epitope sequence, and may comprise sequences which do not contain a universal epitope sequence. Preferably, at least a majority, i.e., 51%, of the amino acid sequence of the peptide comprises a universal epitope sequence. Sequences which do not contribute to a universal epitope sequence can be present at either or both the amino- or carboxyl-terminal end of the peptide. The non-universal epitope sequences preferably are no more than about 10 to about 20 peptidyl residues in toto, and either do not affect the biological activity of the peptide or do not reduce the activity of the peptide by more than about 10 to about 20%.

An epitope peptide of the invention is a peptide that comprises at least about 7 and no more than 40 amino acid residues which are substantially similar or identical to the amino acid sequence of a particular antigen. An epitope peptide of the invention comprises a universal and/or immunodominant epitope sequence. The administration of an epitope peptide of the invention to a mammal results in a mammal that is immunized to the antigen from which the epitope peptide is derived.

A peptide that comprises an amino acid sequence that is “substantially similar” to an amino acid sequence present in an antigen, is a peptide which comprises at least about 7 and no more than about 40, peptidyl residues which have at least about 70%, preferably about 80%, more preferably about 90%, and even more preferably 95%, but less than 100%, contiguous amino acid sequence identity to the amino acid sequence of a particular (native) antigen. The substantially similar peptide of the invention comprises a universal and/or immunodominant epitope sequence. The administration of the peptide results in an immunized mammal.

As used herein, “substantially similar” or “identity” means the proportion of matches between two amino acid sequences. Thus, when sequence homology is given as a percentage, the percentage denotes the proportion of matches over the length of the sequence comparison. Gaps (in either sequence) are permitted to maximize matching. “Homologous” or “substantially similar” indicate less than 100% contiguous amino acid sequence identity to a reference sequence, i.e., the amino acid sequence of a particular antigen.

As used herein, the term “consisting essentially of” with respect to a peptide sequence is defined to mean that at least a majority, i.e., 51%, of the amino acid sequence of the peptide comprises an immunodominant and/or universal epitope sequence.

As used herein, the term “consisting essentially of” with respect to an immuno- or hormono-toxin is defined to mean that the immuno- or hormono-toxin can contain, in addition to fragment A of diphtheria toxin coupled or linked to an antibody or hormone molecule, or a portion thereof which confers binding activity, other agents which do not reduce or impair either the binding or toxin activity of the immuno- or hormono-toxin.

As used herein, the term “CD8+ depleted” or “CD4+ enriched” with respect to a cell population, means that after depletion, the population has fewer CD8+ cells than prior to depletion and/or contains at least about 40 to about 60% of the total number of cells present prior to depletion.

I. The Immune Response

The capacity to respond to immunologic stimuli resides primarily in the cells of the lymphoid system. During embryonic life, a stem cell develops, which differentiates along several different lines. For example, the stem cell may turn into a lymphoid stem cell which may differentiate to form at least two distinct lymphoid populations. One population, called T lymphocytes, is the effector agent in cell-mediated immunity, while the other, called B lymphocytes, is the primary effector of antibody-mediated, or humoral, immunity. The stimulus for B cell antibody production is the attachment of an antigen to B cell surface immunoglobulin. Thus, B cell populations are largely responsible for specific antibody production in the host. For most antigens, B cells require the cooperation of antigen-specific T helper (CD4+) cells for effective production of high affinity antibodies.

Of the classes of T lymphocytes, T helper (Th) or CD4+ cells, are antigen-specific cells that are involved in primary immune recognition and host defense reactions against bacterial, viral, fungi and other antigens. CD4+ cells are necessary to trigger high affinity IgG production from B cells for the vast majority of antigens. The T cytotoxic (Tc) cells are antigen-specific effector cells which can kill target cells following their infection by pathologic agents.

While CD4+ cells are antigen-specific, they cannot recognize free antigen. For recognition and subsequent CD4+ activation and proliferation to occur, the antigen must be processed by suitable cells (antigen presenting cells, APC). APC fragment the antigen molecule and associate the fragments with major histocompatibility complex (MHC) class II products (in humans) present on the APC cell surface. These antigen fragments, or T cell epitopes, are thus presented to receptors or a receptor complex on the CD4+ cell in association with MHC class II products. Thus, CD4+ cell recognition of a pathogenic antigen is MHC class II restricted in that a given population of CD4+ cells must be either autologous or share one or more MHC class II products with the APC. Likewise, Tc cells recognize antigen in association with MHC class I products.

In the case of CD4+ cells, this antigen presenting function is performed by a limited number of APC. It is now well established that CD4+ cells recognize peptides derived from processed soluble antigen in association with class II MHC product, expressed on the surface of macrophages. Recently, other cell types such as resting and activated B cells, dendritic cells, epidermal Langerhans' cells, and human dermal fibroblasts have also been shown to present antigen to CD4+ T cells.

If a given CD4+ cell possesses receptors or a receptor complex which enable it to recognize a given MHC class II product-antigen complex, it becomes activated, proliferates and generates lymphokines, such as interleukin 2 (IL-2). The lymphokines in turn cause the proliferation of several types of “killer” cells, including Tc cells and macrophages, which can exhibit antimicrobial and tumoricidal activity.

After stimulation subsides, survivors of the expanded CD4+ cells remain as member cells in the body, and can expand rapidly again when the same antigen is presented.

Numerous attempts have been made to isolate and maintain homogenous populations of Tc or CD4+ cells and to characterize them in terms of their antigen specificity and MHC restriction. These attempts usually involve the stimulation of mononuclear cells from a seropositive human or murine host with antigenic bacterial or viral preparations in combination with nonproliferative APC, such as irradiated autologous mononuclear cells (MNC). Proliferating polyclonal populations of CD4+ cells or Tc cells can be cloned by limiting dilution to obtain homogenous populations and then further proliferated and characterized by a variety of techniques.

Methods of determining whether PBMCs or lymphoid cells have proliferated, or produced or secreted cytokines, are well known in the art. For example, see Paul, Fundamental Immunology, 3rd ed., Raven Press (1993), and Benjamini et al. (eds.), Immunology: A Short Course, John Wiley & Sons, Inc., 3rd ed. (1996).

II. Indications Amenable to Treatment by the Peptides of the Invention, or Nucleic Acid Molecules Encoding the Peptides of the Invention

The peptides or nucleic acid molecules of the invention are useful to immunize a mammal against an infectious agent or organism. Preferably, these efficacious peptides are recognized by CD4+ cells from a majority of the mammals from a particular species. The peptides are substantially similar or identical in sequence to the amino acid sequence for an antigen of an infectious agent. Thus, a peptide may be selected so as to immunize a mammal against a specific infectious agent, e.g., a virus, bacterium or fungus. Bacteria within the scope of the invention include, but are not limited to, Staphylococcus, Streptococcus (e.g., Streptococcus pneumoniae), Neisseria, Hemophilus, e.g., H. influenza type B, Bordetella such as Bordetella pertussis, Listeria, Erysipelothrix, Corynebacterium, Mycobacterium, Actinomycetes, Enterobacteriaceae, e.g., Salmonella such as Salmonella typhi and Shigella, Vibrionaceae such as Vibrio cholera, Pseudomonas, Yersinia, Francisella, Pasteurella, Actinobacillus, Streptobacillus, Calymmatobacterium, Brucella, Bacillus, Clostridium, Treponema, Borrelia, e.g., Borrelia burgdorferi, Leptospira, Spirillum, Campylobacter, Legionella, Rickettsiae, Bartonella, Chlamydia, and Mycoplasma. Viruses within the scope of the invention include, but are not limited to, Herpesvirus such as herpes simplex, Adenoviruses, Adenovirus-associated Viruses, Human Papovaviruses, Enteroviruses, Orthomyxoviruses, Varicella-Zoster virus, Paramyxoviruses, Pseudomyxoviruses, Rubella, Arboviruses, Rhabdoviruses, Arenaviruses, Hepatitis such as hepatitis A, hepatitis B, and hepatitis C, Rhinoviruses, Coronaviruses, Reoviruses, and Rotaviruses. Other infectious agents within the scope of the invention are associated with systemic mycoses, subcutaneous mycoses, dermatophytosis, and include fungi, as well as parasites such as protozoa and helminths.

Preferred peptides of the invention are those which alone or in combination with adjuvants, are effective to immunize a mammal against mumps, rubella, rabies, small pox, Japanese encephalitis, anthrax, tuberculosis, plague, yellow fever, pneumococcus, typhoid, meningococcus, e.g., (Groups A, C, Y and W-135), cholera, pneumonia, whooping cough, Pneumococcus, Varicella-Zoster virus, measles, polio, tetanus, diphtheria, meningitis, malaria, rotavirus, group A Streptococcus, hepatitis C, Ebola virus and hantavirus.

Preferred peptides from antigens of the genus Plasmodium include, but are not limited to, epitopes specific for each of the four evolutive stages of Plasmodium falciparum, e.g., liver-specific antigen-1 (LSA-1), thromobspondin-related anonymous protein (TRAP), gp190, Pfs25 and Pfs28, which are specific for ookinetes of P. falciparum, Pf155/RESA, GLURP, and MSP-1, which are antigens specific for the blood stage of P. falciparum, and Pfs48/45, SSP-2, Pfs230, Spf66, and PfEMP1; as well as epitopes of other parasites of the genus Plasmodium which infect humans, e.g., P. vivax, P. ovale and P. malariae. Other preferred peptides are those from parasites causing cryptosporidiosis, leishmaniasis, toxoplasmosis and schistosomiasis, e.g., antigens of Schistosoma, such as antigens of S. japonicum, S. mansoni and S. hematobulin, including 9B, 45 kD, 30 kD and 62 kD (see, also, Bergquist, Parasitol. Today, 11, 191 (1995), which is incorporated by reference herein).

Antigens which may be useful for identifying an immunodominant and/or universal epitope specific for M. tuberculosis, include but are not limited to Ag85A, ESAT-6, MPT83, PhoS, hsp65, hsp70, 36 kD, 6 kD, 65 kD, and 85 kD of M. tuberculosis. See, for example Table 1 of Kaufmann et al., Chem. Immunol., 70, 21 (1998), and Lowrie et al., Springer Seminars in Immunology, 19, 161 (1997), both of which are incorporated by reference herein.

Antigens of M. bovis BCG and M. leprae, e.g., the 65 kD, 36 kD, 28 kD or 12 kD protein of M. leprae, or both organisms, may be useful to obtain immunodominant and/or universal epitope peptides.

Hemorrhagic fevers, e.g., Lassa fever, yellow fever, dengue hemorrhagic fever, Kyansanu forest disease, Omsk hemorrhagic fever, Argentine hemorrhagic fever, Bolivian hemorrhagic fever, aseptic lymphocytic choriomeningitis, Rift valley fever, Crimean hemorrhagic fever, and hemorrhagic fever with renal syndrome which includes Korean hemorrhagic fever, epidemic hemorrhagic fever, and nephropathia epidemica, are caused by viruses including Lassa virus, yellow fever virus, dengue virus, Junin virus, Machupo virus, LCM virus, hantaan virus, Marburg virus and Ebola virus. Therefore, preferred peptides of the invention include those having sequences substantially similar to the native antigen(s) of these viruses which are expressed on the surface of these viruses. For example, for hantaviruses, epitope peptides may include sequences from the G1, G2, N, L and/or NS proteins. For Arenaviruses, e.g., Junin virus, epitope peptides can include sequences from the L, Z, N, NP, GP-1 and/or GP-2 protein of an arenavirus associated with hemorrhagic fever. To prepare epitope peptides of the invention for Ebola virus, sequences from VP35, VP40, NP, GP, VP24, VP30, and/or L protein may be screened by methods described herein.

III. Identification of an Epitope Peptide Falling within the Scope of the Invention

The identification of a universal and/or immunodominant epitope sequence in an antigen permits the development and use of a peptide-based immunogen. The administration of epitope peptides which contain a universal and/or immunodominant epitope sequence can induce an immunizing effect in many, if not all, mammals of a particular species, preferably those of differing immune response haplotypes. Moreover, the use of peptide immunogens is less likely to produce the undesirable side effects associated with the use of the full-length antigen. These epitope peptides can be identified by in vitro and in vivo assays, such as the assays described hereinbelow (see, for example, Conti-Fine et al., 1997; and Wang et al., 1997). It is recognized that not all peptides falling within the scope of the invention may result in immunization, or result in the same degree of immunization.

To identify epitope peptides useful to immunize a mammal, the infectious agent which is associated with an indication, pathology or disease is identified. In order to prepare an immunodominant peptide to immunize a host organism against a newly recognized or uncharacterized infectious agent, the antigen(s) of the infectious agent that are recognized by the immune system are identified. For example, the inactivated infectious agent is administered to an organism, e.g., a mouse. Subsequently, the serum and T cells of that organism are collected. Alternatively, serum and T cells are collected from a mammal exposed to the infectious agent and/or a mammal having manifestations of the indication, pathology or disease.

The serum is employed to determine which antigen(s) of the infectious agent are recognized by antibodies, e.g., using Western blot. Bands that are strongly recognized by antibodies are excised and the protein in the band purified. T cells from the immunized organism or human donor are mixed with the purified protein and the proliferative response of the T cells measured. If there is a vigorous T cell response, the protein is subjected to sequencing. The amino acid sequence is used to design primers that can amplify the nucleic acid sequence which encodes the protein. The full length nucleic acid sequence is translated, and overlapping peptides of the encoded protein are prepared and screened as described below, i.e., using human PBMC or T cell lines from individuals exposed to the organism or vaccinated with the organism.

If the entire amino acid sequence of the polypeptide(s) encoded by the genome or nucleic acid of, or associated with, the agent, and which are expressed on the pathogen's surface and/or which are recognized by immune cells of a host organism, is known, then 20 amino acid residue peptides are obtained or prepared which span the entire amino acid sequence of the polypeptide and which overlap the adjacent peptide by 5-10 residues. In this manner, a peptide may include sequences which correspond to a portion of a universal and/or immunodominant epitope sequence. For example, human PBMC or T cells lines obtained from individuals that had been exposed to the infectious agent and/or which manifested symptoms associated with the indication, pathology or disease caused by the infectious agent, can be used to identify whether a peptide corresponding to a region of the antigen(s) comprises an immunodominant and/or universal epitope peptide.

Alternatively, or in addition to the use of human PBMC or T cell lines, animal models can be employed to determine whether a particular peptide comprises an immunodominant and/or universal epitope sequence. For example, a peptide is administered to HuPBL-SCID mice (Conti-Fine et al., Anal. N.Y. Acad. Sci., 841, 283 (1998)) or murine MHC knockout, human class II transgenic mice (Raju et al., Anal. N.Y. Acad. Sci., 841, 360 (1998)) and the immune response measured by methods well known to the art. For HuPBL-SCID mice, the mice can also be reconstituted with PBLs from a human subject that was exposed to a particular infectious agent, antigen or peptide thereof. Immunodominant peptides are those which elicit a strong immune response. A series of HuPBL-SCID mice or murine MHC knockout, human class II transgenic mice, where each one of the series has a different genotype at the class II loci relative to the other members of the series, may be employed to identify universal epitope sequences.

The portion of the antigen which is processed by immune cells or by proteases may also be identified. One method to identify processed portions of an antigen is to expose antigen presenting cells from at least one human to antigen. MHC class II molecules are then purified and the peptides bound thereto released and sequenced, preferably by mass spectroscopy.

These peptides may be individually screened in vitro, e.g., for binding to class II molecules, ability to induce T cell proliferation or secretion of at least one cytokine (Th1 cytokines include IFN-γ, IL-12 and IL-2, and Th2 cytokines include IL-4, IL-5 and IL-10) of CD4+ cell lines specific for the antigen having the peptide sequence, isolated CD4+ cells, CD8+ depleted spleen cells or lymph node cells, or CD8+ depleted peripheral blood mononuclear cells (PBMC) (Manfredi et al., Anal. Biochem., 211, 267 (1993); Yuen et al., J. Autoimmun., 9, 67 (1996); Manfredi et al., J. Immunol., 4165 (1994)). The cell populations listed above are obtained from an experimental animal or human subject that had been exposed to the infectious agent or the antigen. An immunospot ELISA or other biological assay is employed to determine the cytokine which is secreted after the peptide is added to the culture (see, for example, Wang et al., Neurol., 50, 1045 (1998); Wang et al., Neurol., 48 1643 (1997)).

IV. Preparation of the Peptides of the Invention

A. Nucleic Acid Molecules of the Invention

1. Sources of the Nucleic Acid Molecules of the Invention

Sources of nucleotide sequences from which a nucleic acid molecule encoding a peptide of the invention include RNA or DNA from any infectious agent. Other sources of DNA molecules of the invention include libraries derived from the nucleic acid of the genome of any infectious agent. An example of an isolated nucleic acid molecule of the invention is RNA or DNA that encodes at least a portion of an antigen of an infectious agent, and shares at least about 80%, preferably at least about 90%, and more preferably at least about 95%, contiguous nucleotide sequence identity to the native nucleic acid sequence encoding that antigen.

Moreover, the present DNA molecules may be prepared in vitro, e.g., by synthesizing an oligonucleotide of about 100, preferably about 75, more preferably about 50, and even more preferably about 40, nucleotides in length, or by subcloning a portion of a DNA segment that encodes a particular peptide.

2. Isolation of a Gene Encoding a Peptide of the Invention

A nucleic acid molecule encoding a peptide of the invention can be identified and isolated using standard methods, as described by Sambrook et al., Molecular Cloning: A Laboratory Manual, Cold Spring Harbor, N.Y. (1989). For example, reverse-transcriptase PCR (RT-PCR) can be employed to isolate and clone a preselected cDNA. Oligo-dT can be employed as a primer in a reverse transcriptase reaction to prepare first-strand cDNAs from isolated RNA which contains RNA sequences of interest, e.g., total RNA isolated from infected mammalian tissue. RNA can be isolated by methods known to the art, e.g., using TRIZOL™ reagent (GIBCO-BRL/Life Technologies, Gaithersburg, Md.). Resultant first-strand cDNAs are then amplified in PCR reactions.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers comprising at least 7-8 nucleotides. These primers will be identical or similar in sequence to opposite strands of the template to be amplified. PCR can be used to amplify specific RNA sequences, specific DNA sequences from total DNA, and cDNA transcribed from total cellular RNA, bacteriophage or plasmid sequences, and the like. See generally Mullis et al., Cold Spring Harbor Symp. Quant. Biol., 51, 263 (1987); Erlich, ed., PCR Technology, (Stockton Press, NY, 1989). Thus, PCR-based cloning approaches rely upon conserved sequences deduced from alignments of related gene or polypeptide sequences.

Primers are made to correspond to highly conserved regions of polypeptides or nucleotide sequences which were identified and compared to generate the primers, e.g., by a sequence comparison of genes related to a encoding a polypeptide of an infectious agent. One primer is prepared which is predicted to anneal to the antisense strand, and another primer prepared which is predicted to anneal to the sense strand, of a nucleic acid molecule which encodes the preselected peptide.

The products of each PCR reaction are separated via an agarose gel and all consistently amplified products are gel-purified and cloned directly into a suitable vector, such as a known plasmid vector. The resultant plasmids are subjected to restriction endonuclease and dideoxy sequencing of double-stranded plasmid DNAs. Alternatively, isolated gel-purified fragments may be directly sequenced.

As used herein, the terms “isolated and/or purified” refer to in vitro isolation of a DNA, peptide or polypeptide molecule from its natural cellular environment, and from association with other components of the cell, such as nucleic acid or polypeptide, so that it can be sequenced, replicated, and/or expressed. For example, an “isolated, preselected nucleic acid” is RNA or DNA containing greater than 9, preferably 36, and more preferably 45 or more, sequential nucleotide bases that encode at least a portion of a peptide of the invention, or a variant thereof, or a RNA or DNA complementary thereto, that is complementary or hybridizes, respectively, to RNA or DNA encoding the peptide, or polypeptide having said peptide, and remains stably bound under stringent conditions, as defined by methods well known in the art, e.g., in Sambrook et al., supra. Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell.

As used herein, the term “recombinant nucleic acid” or “preselected nucleic acid,” e.g., “recombinant DNA sequence or segment” or “preselected DNA sequence or segment” refers to a nucleic acid, e.g., to DNA, that has been derived or isolated from any appropriate source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. See Lawn et al., Nucleic Acids Res., 9, 6103 (1981), and Goeddel et al., Nucleic Acids Res., 8, 4057 (1980). Therefore, “preselected DNA” includes completely synthetic DNA sequences, semi-synthetic DNA sequences, DNA sequences isolated from biological sources, and DNA sequences derived from RNA, as well as mixtures thereof.

As used herein, the term “derived” with respect to a RNA molecule means that the RNA molecule has complementary sequence identity to a particular DNA molecule.

3. Variants of the Nucleic Acid Molecules of the Invention

Nucleic acid molecules encoding amino acid sequence variants of a peptide of the invention are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the preselected peptide.

Oligonucleotide-mediated mutagenesis is a preferred method for preparing amino acid substitution variants of a peptide. This technique is well known in the art as described by Adelman et al., DNA, 2, 183 (1983). Briefly, DNA is altered by hybridizing an oligonucleotide encoding the desired mutation to a DNA template, where the template is the single-stranded form of a plasmid or bacteriophage containing the unaltered or native DNA sequence. After hybridization, a DNA polymerase is used to synthesize an entire second complementary strand of the template that will thus incorporate the oligonucleotide primer, and will code for the selected alteration in the preselected DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. U.S.A., 75, 5765 (1978).

The DNA template can be generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153, 3 (1987). Thus, the DNA that is to be mutated may be inserted into one of these vectors to generate single-stranded template. Production of the single-stranded template is described in Sections 4.21-4.41 of Sambrook et al., Molecular Cloning: A Laboratory Manual (Cold Spring Harbor Laboratory Press, N.Y. 1989).

Alternatively, single-stranded DNA template may be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase 1, is then added to synthesize the complementary strand of the template using the oligonucleotide as a primer for synthesis. A heteroduplex molecule is thus formed such that one strand of DNA encodes the mutated form of the peptide, and the other strand (the original template) encodes the native, unaltered sequence of the peptide. This heteroduplex molecule is then transformed into a suitable host cell, usually a prokaryote such as E. coli JM101. After the cells are grown, they are plated onto agarose plates and screened using the oligonucleotide primer radiolabeled with 32-phosphate to identify the bacterial colonies that contain the mutated DNA. The mutated region is then removed and placed in an appropriate vector for peptide or polypeptide production, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(αS) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(αS) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101.

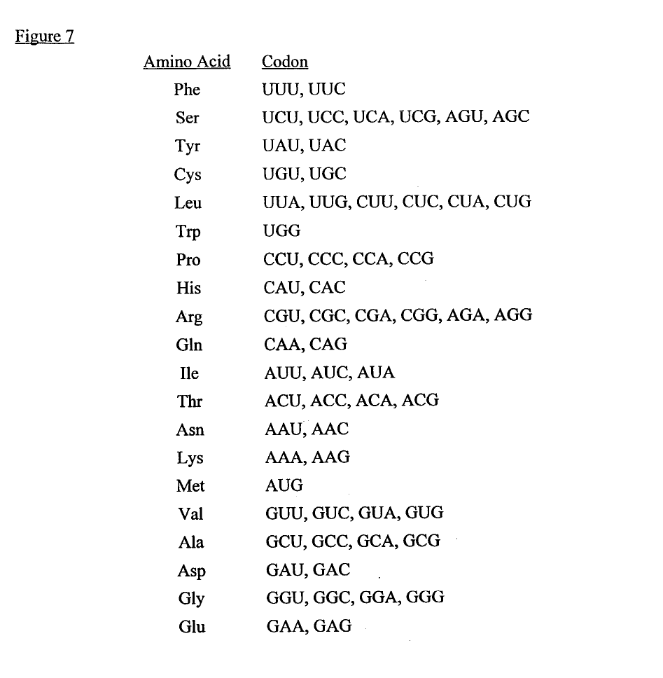

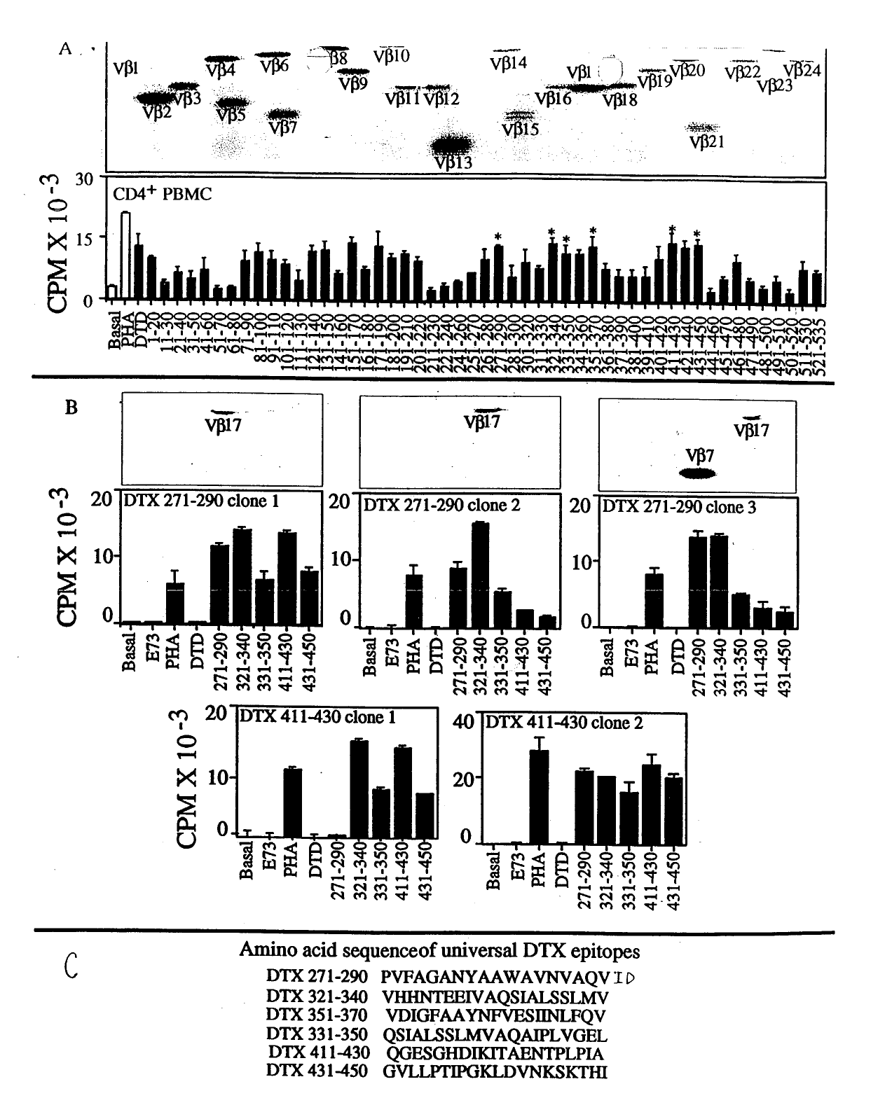

For example, a preferred embodiment of the invention is an isolated and purified DNA molecule comprising a preselected DNA segment encoding a peptide of the invention, which includes a DNA segment that has a nucleotide substitution which is “silent” (see FIG. 7). That is, when silent nucleotide substitutions are present in a codon, the same amino acid is encoded by the codon with the nucleotide substitution as is encoded by the codon without the substitution. For example, if a peptide of the invention includes leucine, leucine is encoded by the codon CTT, CTC, CTA and CTG. Thus, if the third codon in the DNA segment is CTC, the same peptide is encoded by a DNA segment having CTT, CTA or CTG for CTC at that position. Other “silent” nucleotide substitutions can be ascertained by reference to FIG. 9 and page D1 in Appendix D in Sambrook et al., Molecular Cloning: A Laboratory Manual (1989). Nucleotide substitutions can be introduced into DNA segments by methods well known to the art. See, for example, Sambrook et al., supra. Likewise, nucleic acid molecules encoding other antigens or peptides of infectious agents may be modified in a similar manner, so as to yield nucleic acid molecules of the invention having silent nucleotide substitutions, or to yield nucleic acid molecules having nucleotide substitutions that result in amino acid substitutions (see peptide variants hereinbelow).

4. Chimeric Expression Cassettes

To prepare expression cassettes for transformation herein, the recombinant or preselected DNA sequence or segment may be circular or linear, double-stranded or single-stranded. Generally, the preselected DNA sequence or segment is in the form of chimeric DNA, such as plasmid DNA, that can also contain coding regions flanked by control sequences which promote the expression of the preselected DNA present in the resultant cell line.

As used herein, “chimeric” means that a vector comprises DNA from at least two different species, or comprises DNA from the same species, which is linked or associated in a manner which does not occur in the “native” or wild type of the species.

Aside from preselected DNA sequences that serve as transcription units for a peptide, or portions thereof, a portion of the preselected DNA may be untranscribed, serving a regulatory or a structural function. For example, the preselected DNA may itself comprise a promoter that is active in mammalian cells, or may utilize a promoter already present in the genome that is the transformation target. Such promoters include the CMV promoter, as well as the SV40 late promoter and retroviral LTRs (long terminal repeat elements), although many other promoter elements well known to the art may be employed in the practice of the invention.

Other elements functional in the host cells, such as introns, enhancers, polyadenylation sequences and the like, may also be a part of the preselected DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a peptide or polypeptide if it is expressed as a preprotein that participates in the secretion of the peptide or polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The preselected DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt dhfr, bar, aroA, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein. For example, J. Sambrook et al., Molecular Cloning: A Laboratory Manual, Cold Spring Harbor Laboratory Press (2d ed., 1989), provides suitable methods of construction.

5. Transformation into Host Cells

The recombinant DNA can be readily introduced into the host cells, e.g., mammalian, bacterial, yeast or insect cells by transfection with an expression vector comprising DNA encoding a preselected peptide by any procedure useful for the introduction into a particular cell, e.g., physical or biological methods, to yield a transformed cell having the recombinant DNA stably integrated into its genome, so that the DNA molecules, sequences, or segments, of the present invention are expressed by the host cell.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For mammalian gene therapy, it is desirable to use an efficient means of precisely inserting a single copy gene into the host genome. Viral vectors, and especially retroviral vectors, have become the most widely used method for inserting genes into mammalian, e.g., human cells. Other viral vectors can be derived from poxviruses, herpes simplex virus 1, adenoviruses and adeno-associated viruses, and the like. See, for example, U.S. Pat. Nos. 5,350,674 and 5,585,362.

As used herein, the term “cell line” or “host cell” is intended to refer to well-characterized homogenous, biologically pure populations of cells. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources.

“Transfected” or “transformed” is used herein to include any host cell or cell line, the genome of which has been altered or augmented by the presence of at least one preselected DNA sequence, which DNA is also referred to in the art of genetic engineering as “heterologous DNA,” “recombinant DNA,” “exogenous DNA,” “genetically engineered,” “non-native,” or “foreign DNA,” wherein said DNA was isolated and introduced into the genome of the host cell or cell line by the process of genetic engineering. The host cells of the present invention are typically produced by transfection with a DNA sequence in a plasmid expression vector, a viral expression vector, or as an isolated linear DNA sequence. Preferably, the transfected DNA is a chromosomally integrated recombinant DNA sequence, which comprises a gene encoding the peptide.

To confirm the presence of the preselected DNA sequence in the host cell, a variety of assays may be preformed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described hereinabove to identify agents falling within the scope of the invention.

To detect and quantitate RNA produced from introduced preselected DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the peptide products of the introduced preselected DNA sequences or evaluating the phenotypic changes brought about by the expression of the introduced preselected DNA segment in the host cell.

B. Peptides, Peptide Variants, and Derivatives Thereof

The present isolated, purified peptides or variants thereof (i.e., peptides that are substantially similar to a reference peptide), can be synthesized in vitro, e.g., by the solid phase peptide synthetic method or by recombinant DNA approaches (see above). The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; and Bavaay and Merrifield, “The Peptides,” eds. F. Gross and F. Meienhofer, Vol. 2 (Academic Press, 1980) pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, of a given peptide can be readily prepared. For example, amides of the peptide or peptide variants of the present invention may also be prepared by techniques well known in the art for converting a carboxylic acid group or precursor to an amide. A preferred method for amide formation at the C-terminal carboxyl group is to cleave the peptide from a solid support with an appropriate amine, or to cleave in the presence of an alcohol, yielding an ester, followed by aminolysis with the desired amine.

Salts of carboxyl groups of a peptide or peptide variant of the invention may be prepared in the usual manner by contacting the peptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide or peptide variants may be prepared by utilizing an N-acyl protected amino acid for the final condensation, or by acylating a protected or unprotected peptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the peptide or peptide variant. Other amino-terminal modifications include aminooxypentane modifications (see Simmons et al., Science, 276, 276 (1997)).

In addition, the amino acid sequence of a peptide can be modified so as to result in a peptide variant (see above). The modification includes the substitution of at least one amino acid residue in the peptide for another amino acid residue, including substitutions which utilize the D rather than L form, as well as other well known amino acid analogs. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, and tert-butylglycine.

One or more of the residues of the peptide can be altered, so long as the peptide variant is biologically active. For example, it is preferred that the variant has at least about 10% of the biological activity of the corresponding non-variant peptide. Conservative amino acid substitutions are preferred—that is, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids.

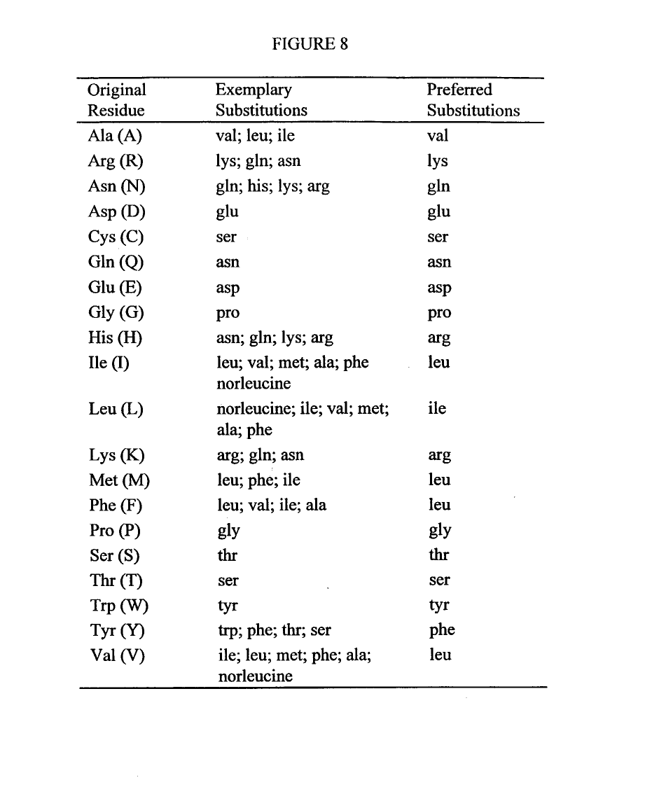

Conservative substitutions are shown in FIG. 8 under the heading of exemplary substitutions. More preferred substitutions are under the heading of preferred substitutions. After the substitutions are introduced, the variants are screened for biological activity.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; tip, tyr, phe.

The invention also envisions peptide variants with non-conservative substitutions. Non-conservative substitutions entail exchanging a member of one of the classes described above for another.

Acid addition salts of the peptide or variant peptide, or of amino residues of the peptide or variant peptide, may be prepared by contacting the peptide or amine with one or more equivalents of the desired inorganic or organic acid, such as, for example, hydrochloric acid. Esters of carboxyl groups of the peptides may also be prepared by any of the usual methods known in the art.

V. Dosages, Formulations and Routes of Administration of the Peptides of the Invention

The peptides or nucleic acid molecules of the invention, including their salts, are preferably administered so as to result in a protective immune response, a reduction in at least one symptom associated with infection of the host by the infectious agent, and/or an increase in the amount of antibody specific for the administered peptide or infectious agent. To achieve this effect(s), the peptide, a variant thereof or a combination thereof, agent may be administered at dosages of at least about 0.001 to about 100 mg/kg, more preferably about 0.01 to about 10 mg/kg, and even more preferably about 0.1 to about 5 mg/kg, of body weight, although other dosages may provide beneficial results. The amount administered will vary depending on various factors including, but not limited to, the peptide(s) chosen, the infectious agent, the weight, the physical condition, and the age of the mammal, and whether prevention or treatment is to be achieved. Such factors can be readily determined by the clinician employing animal models or other test systems which are well known to the art.

Administration of sense nucleic acid molecule may be accomplished through the introduction of cells transformed with an expression cassette comprising the nucleic acid molecule (see, for example, WO 93/02556) or the administration of the nucleic acid molecule (see, for example, Felgner et al., U.S. Pat. No. 5,580,859, Pardoll et al., Immunity, 3, 165 (1995); Stevenson et al., Immunol. Rev., 145, 211 (1995); Molling, J. Mol. Med., 75, 242 (1997); Donnelly et al., Ann. N.Y. Acad. Sci., 772, 40 (1995); Yang et al., Mol. Med. Today, 2, 476 (1996); Abdallah et al., Biol. Cell, 85, 1 (1995)). Pharmaceutical formulations, dosages and routes of administration for nucleic acids are generally disclosed, for example, in Felgner et al., supra.

Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

To prepare the composition, peptides are synthesized or otherwise obtained, purified and then lyophilized and stabilized. The peptide can then be adjusted to the appropriate concentration, and optionally combined with other agents. For example, a peptide having an immunodominant and/or a universal epitope sequence may be administered alone, with other peptides that also have an immunodominant and/or a universal epitope sequence, and/or in combination with the inactivated infectious agent or other adjuvants, e.g., aluminum hydroxide, dimethyl dioctadecylammonium bromide, Quil-A Saponin, QS-21 and monophosporyl lipid A. See also, Vogel et al., A compendium of vaccines adjuvants. In: Vaccine Design: The Subunit and Adjuvant Approach, Powell et al. (Eds), Plenum Press, NY, pp. 141-228 (1995), in an amount that results in protective or enhanced immune response.

The absolute weight of a given peptide included in a unit dose of a vaccine can vary widely. For example, about 0.01 to about 10 mg, preferably about 0.5 to about 5 mg, of at least one peptide of the invention, and preferably a plurality of peptides specific for a particular antigen, each containing a universal and/or immunodominant epitope sequence, can be administered.

Thus, one or more suitable unit dosage forms comprising the therapeutic agents of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including oral, or parenteral, including by rectal, transdermal, subcutaneous, intravenous, intramuscular, intraperitoneal, intrathoracic, intrapulmonary and intranasal (respiratory) routes. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are preferably combined with a pharmaceutically acceptable carrier, diluent or excipient to form a pharmaceutical formulation, or unit dosage form. Preferably, orally administered therapeutic agents of the invention are formulated for sustained release, e.g., the agents are microencapsulated. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Pharmaceutical formulations containing the therapeutic agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and α-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles or or waxes. These coatings, envelopes, and protective matrices are useful to coat indwelling devices, e.g., stents, catheters, peritoneal dialysis tubing, and the like. Preferably, the peptides are formulated as microspheres or nanospheres.

The therapeutic agents of the invention can be delivered via patches for transdermal administration. See U.S. Pat. No. 5,560,922 for examples of patches suitable for transdermal delivery of a therapeutic agent. Patches for transdermal delivery can comprise a backing layer and a polymer matrix which has dispersed or dissolved therein a therapeutic agent, along with one or more skin permeation enhancers. The backing layer can be made of any suitable material which is impermeable to the therapeutic agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which therapeutic agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the therapeutic agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydro-gel), polyvinylidene chloride, poly(ethylene terephthalate), butyl rubber, epichlorohydrin rubbers, ethylenvinyl alcohol copolymers, ethylene-vinyloxyethanol copolymers; silicone copolymers, for example, polysiloxane-polycarbonate copolymers, polysiloxanepolyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the therapeutic agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Therapeutic agents released from a transdermal delivery system must be capable of penetrating each layer of skin. In order to increase the rate of permeation of a therapeutic agent, a transdermal drug delivery system must be able in particular to increase the permeability of the outermost layer of skin, the stratum corneum, which provides the most resistance to the penetration of molecules. The fabrication of patches for transdermal delivery of therapeutic agents is well known to the art.

For topical administration, the therapeutic agents may be formulated as is known in the art for direct application to a target area. Conventional forms for this purpose include wound dressings, coated bandages or other polymer coverings, ointments, creams, lotions, pastes, jellies, sprays, and aerosols. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

Preferably, the peptide or nucleic acid of the invention is administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific indication or disease. Any statistically significant attenuation of one or more symptoms of an indication or disease that has been treated pursuant to the method of the present invention is considered to be a treatment of such indication or disease within the scope of the invention.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0. saline solutions and water.

The agents of the present invention can be administered as a dry powder or in an aqueous solution. Preferred aerosol pharmaceutical formulations may comprise, for example, a physiologically acceptable buffered saline solution containing between about 0.1 mg/ml and about 100 mg/ml of one or more of the agents of the present invention specific for the indication or disease to be treated.

Dry aerosol in the form of finely divided solid peptide or nucleic acid particles that are not dissolved or suspended in a liquid are also useful in the practice of the present invention. Peptide or nucleic acid may be in the form of dusting powders and comprise finely divided particles having an average particle size of between about 1 and 5 μm, preferably between 2 and 3 μm. Finely divided particles may be prepared by pulverization and screen filtration using techniques well known in the art. The particles may be administered by inhaling a predetermined quantity of the finely divided material, which can be in the form of a powder.

For administration to the upper (nasal) or lower respiratory tract by inhalation, the therapeutic agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.).

For intra-nasal administration, the therapeutic agent may be administered via nose drops, a liquid spray, such as via a plastic bottle atomizer or metered-dose inhaler. Typical of atomizers are the Mistometer (Wintrop) and the Medihaler (Riker).

The formulations and compositions described herein may also contain other ingredients such as antimicrobial agents, or preservatives. Furthermore, the active ingredients may also be used in combination with other therapeutic agents, for example, bronchodilators.

Preferred delivery systems for a peptide can include coupling the peptide to a carrier or an intact attenuated microbe, such as an inactivated virus or attenuated bacterium, e.g., weakened Salmonella, preparing a multiple antigen peptide, using liposomes or other immunostimulating complexes. Preferably, the delivery system enhances the immunogenicity of the peptide. Preferred carrier proteins include large antigenic proteins such as DTD and TTD, or a fusion protein having a carrier protein of bacterial, e.g., Salmonella flagellin, or viral origin. Viral vectors that may be employed to deliver nucleic acid encoding the peptide include, but are not limited to, retroviral vectors, vaccinia virus vectors, adenovirus vectors or canarypox virus vectors.

The invention will be described with reference to the following non-limiting examples.

### EXAMPLE 1

DTX is a good antigen to use to study immune recognition in humans, because most individuals are immunized against this antigen, and the three-dimensional structure of DTX is known (Chol et al., Nature, 357, 216 (1992); Bennett et al., Protein Sci., 3, (1994); Bennett et al., Protein Sci., 3, 1464 (1994)). As discussed above, existing anti-diphtheria vaccines frequently produce undesirable side effects of differing severity, especially in adults. While these side effects are reduced when a low dose is used, a single low dose is not effective in inducing protective antibody titers.

The identification of an IRS in DTX, described hereinbelow, permits the development and use of a peptide-based or peptide-enhanced vaccine to DTX. DTX-specific peptides which contain an IRS can induce an immune response in many, if not all, individuals, regardless of HLA haplotype. Moreover, such vaccines will not produce the undesirable side effects associated with the contaminants present in the anti-diphtheria vaccines currently in use because the vaccines lack material currently employed in diphtheria vaccines, i.e., they are peptide-based vaccines, or only contain these materials in low amounts, i.e., they are peptide-enhanced vaccines. Thus, at least one DTX-specific peptide containing an IRS, where the peptide is of sufficient length to induce a B cell response, can be administered as the active component of an anti-DTX vaccine. A more preferred embodiment of the invention is the administration of a vaccine comprising a plurality of DTX-specific peptides each containing an IRS, wherein each peptide is of sufficient length to trigger a B cell response.

To prepare the vaccine, peptides would by synthesized or otherwise obtained and then lyophilized and stabilized. The peptide can then be adjusted to the appropriate concentration, and optionally combined with other agents. The absolute weight of a given peptide included in a unit dose of a vaccine can vary widely. For example, 0.5-10 mg, preferably 1-5 mg, of at least one DTX-specific peptide, and preferably a plurality of DTX-specific peptides, containing an IRS, can be administered. The dose administered can depend upon factors such as the weight, age, and physical condition of the mammal to be immunized. Such factors can be readily determined by the clinician employing animal models or other test systems which are well known to the art. A unit dose of the vaccine is preferably administered parenterally, e.g., by subcutaneous or by intramuscular injection.

A preferred embodiment of the invention is an enhanced DTX-specific peptide-based vaccine which comprises at least one DTX-specific peptide containing an IRS and an amount of DTD which is sufficient to induce an antibody response. Administration of synthetic peptides of a given protein antigen containing CD4+ T cell epitopes can potentiate the immune response to the antigen, i.e., DTD, given concomitantly or subsequently to the synthetic peptide epitope. Thus, the concomitant administration of a low dose of DTD with at least one DTX-specific peptide containing an IRS results in minimal undesirable side effects while stimulating both B and CD4+ cells to produce an effective immune response. For example, such an enhanced peptide-based vaccine can include 1 unit of DTD plus 0.5-10 mg, preferably 1-5 mg, of at least one, preferably a plurality of, DTX-specific peptide containing an IRS. In infants, such a vaccine would follow the customary dosing schedule. In adults, a single dose of the enhanced vaccine may be needed every 3-5 years.

Either of these two embodiments produce a vaccine that is equally inexpensive and efficacious as the vaccines currently in use, but reduce or eliminate the side effects associated with the currently employed vaccines.

Overlapping synthetic peptides spanning the entire DTX sequence were employed to identify sequence regions recognized by CD4+ cells of seven healthy humans with different HLA haplotypes.

Materials and Methods

Subjects. Table 1 summarizes some salient features of the seven subjects studied. These patients had been immunized with DTD, and boosted either immediately prior to this study or in the recent past.

TABLE 1

| Subject # | Age, Sex | HLA-DR, DQ haplotype |
|---|---|---|
| 1. | 33, M | DR8(w8), DQw7(w3) |
| | | DR6(w14), DRw52c, DQw5(w1) |
| 2. | 44, F | DR5(w11), DRw52, DQw2 |
| | | DR4, DRw53, DQw7(w3) |
| 3. | 31, M | DR4, DRw53, DQw5(w1) |
| | | DR10(w10), DQw4 |
| 4. | 27, M | DR4(Dw10), DRw53, DQw8(w3) |
| | | DR2(w15), Dw2, DRw51, DQw6(w1) |
| 5. | 27, M | DR4, DRw53, DQw7(w3) |
| | | DR5(w12), DRw52a |
| 6. | 25, F | DR1, DQw5(w1) |
| | | DR2(w15), DRw51(Dw2), DQw6(w1) |
| 7. | 27, M | DR5(w12), DRw52c, DQw8(w3) |
| | | DR6(w13), DRw52, DQw6(w1) |

a Determined by restriction fragment length polymorphism (Noreen et al., Transp. Proc., 21 2968 (1989)) and/or oligonucleotide typing (Kumura et al., In: Proceedings of the Eleventh International Histocompatibility Workshop and Conference: HLA, Oxford University Press, p. 397 (1992)).

Peptide synthesis. 53 peptides, 20 residues long and overlapping each other by 10 residues were synthesized. In sum, the peptides corresponded to the complete DTX sequence (Heighten et al., PNAS USA, 82, 7048 (1988)). The length of the peptides was chosen because, although class II restricted epitopes are only 13-17 residues in length, the presence of extra residues does not interfere with epitope presentation, as the binding groove of DR molecule is open-ended on both sides (Stern et al., Nature, 368, 215 (1994)). The sequence overlap is close to the length of class II restricted T epitopes so as to reduce the chance of missing epitopes “broken” between peptides. Each peptide is numerically designated with a code which includes two numbers, referring to the position on the DTX sequence of the first and the last peptide residues.

The amino acid composition of the peptides found to contain IRSs was verified by phenylisothiocyanate derivatization of amino acid residues released by acid hydrolysis, followed by separation by reverse-phase HPLC (Heinrickson et al., Anal. Biochem., 136, 65 (1993)). The results of the composition analysis corresponded with the expected theoretical values. Consistent results were obtained for different batches of the same peptide sequence.

The molecular weight of peptides with IRSs was verified by mass spectrometry. For all peptides a major peak of the expected molecular weight was present.

CD8+ T cell Depletion and Proliferation Assay. CD8+ T cells can inhibit the in vitro response of human CD4+ cells to Ags (Protti et al., J. Immunol., 144, 1276 (1990); Manfredi et al., J. Clin. Invest., 92, 1055 (1993)). Thus, the results of proliferation assays carried out with populations containing both CD4+ and CD8+ cells may be difficult to interpret. To identify a DTX peptide sequence recognized by CD4+ T cells in the peripheral blood, PBMC were depleted of CD8+ cells by paramagnetic beads. Yields of the CD8+ depleted, CD4+ enriched cell population (referred to as either CD4+ enriched or CD8+ depleted cells) were consistently 45=55% of the starting PBMC population.

CD4+ enriched cells, diluted to 1×10⁶/ml in 1640 (Gibco, Grand Island, N.Y.) with 10% heat inactivated AB human serum, 2 mM L-glutamine, 100 U/ml penicillin and 50 μg/ml streptomycin (Tissue Culture Medium, TCM), were plated in triplicate in 96 round bottom well plates, and stimulated with each one of the following: phytohemagglutinin (PHA, 10 μg/ml, Wellcome, London, UK), interleukin 2 (IL-2, Lymphocult, Biotest Diagnostic Inc., Dreieich, Germany; final concentration of 10 U/ml), DTD (Wyeth Laboratories, Inc., PA; 10 μg/ml), or an individual synthetic peptide. Basal growth rate was determined from triplicate wells containing CD4+ enriched cells cultivated without any stimulus. After five days, the cultures were pulsed for 16 hours with ³H-thymidine (1 μCi per well, specific activity 6.7 Ci/mmol, Amersham, Arlington Heights, Ill.), collected with a Titertek multiple harvester (Skatron Inc., Sterling, Va.), and the ³H-thymidine incorporation was measured by liquid scintillation.

Propagation of CD4+ cell lines specific for DTD and proliferation assay. PBMC were suspended (1-2×10⁶ cell/ml) in TCM containing 10 μg/ml DTD, and cultivated in T25 flasks (Costar, Cambridge, Mass.) for 1 week. The reactive lymphoblasts were isolated on Percoll gradients, expanded in TCM containing T-cell growth factor (TCGF, Lymphocult, Biotest Diagnostic, Dreieich, Germany, at a final concentration of IL-2 of 10 units/ml), and enriched in DTD-specific cells by weekly stimulations with the same amount of DTD plus irradiated (4,000 rads: 1 rad=0.01 Gy) autologous PBMC as APC. The response to DTD and PHA of the T cell lines obtained was tested weekly.

Proliferation assays with CD4+ cell lines for DTD. Proliferation assays were carried out with CD4+ lines, using 2×10⁴ cells/well, irradiated autologous PBMC (2×10⁵ cells/well) as APC, and the Ags described above for CD4+ enriched PBMC. Basal growth rate (Blank) was determined from triplicate wells containing CD4+ cell lines cultivated without any stimulus. After one day, the cultured cells were pulsed for 16 hours with ³H-thymidine, collected, and the ³H-thymidine incorporation measured as described above for CD4+ enriched PBMC.

Flow cytometry. The phenotype of the T cell lines and of the CD4+ enriched PBMC was determined using a FACStar^{R} cell sorter (Becton Dickinson and Co., Mountain View, Calif.) and phycoerythrin-conjugated Leu 4 (anti-CD3), and FITC-conjugated Leu 2 (anti-CD8) and Leu 3 (anti-CD4) antibodies (Becton Dickinson, San Jose, Calif.), as described by Mojola et al., J. Clin. Invest., 93, 1020 (1994)).

HLA class II restriction of CD4+ recognition of DTX IRSs. The DR, DP, or DQ restriction of the IRSs recognized by the anti-DTD CD4+ lines was investigated for all the lines in inhibition experiments, using commercially available purified anti-DR, anti-DP and anti-DQ mAbs (Becton Dickinson, San Jose, Calif.), as described by Mojola et al. (J. Immunol., 1521, 4686 (1994)).

Results

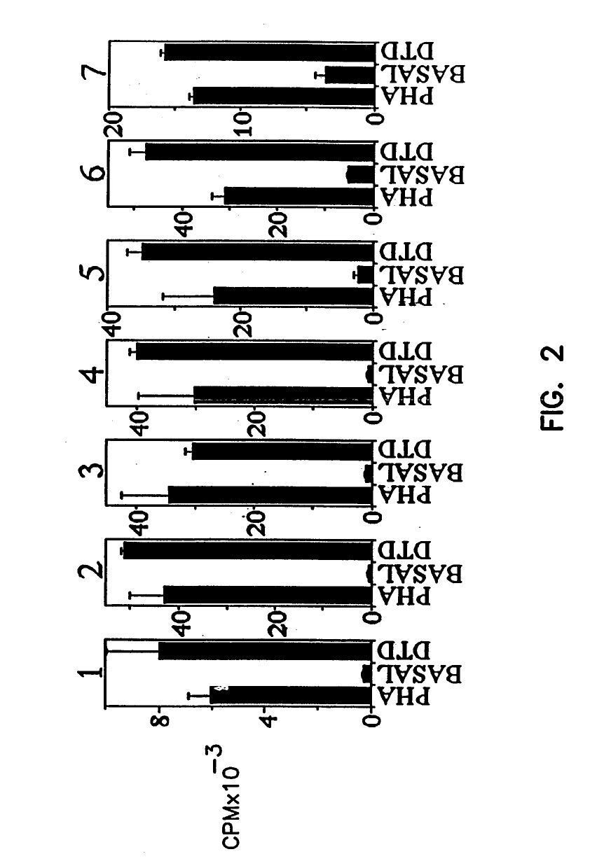

Propagation and characterization of anti-DTD T cell lines from healthy subjects. Anti-DTD T cell lines were successfully obtained from all the subjects tested. The lines were considered sufficiently enriched in anti-DTD T cells when their response to DTD in proliferation assays was comparable to, or better than, that of PHA. This occurred after 3-4 cycles of stimulation with DTD. The lines were predominantly or exclusively CD3+, CD4+, CD8− (Table 2). The results of one representative experiment for each line, testing the response to PHA and DTD, are shown in FIG. 2.

TABLE 2

| T cell line | CD3 + cells | CD3 + CD4 + | CD3 + CD8 + |
|---|---|---|---|
| (Subject #) | (%) | cells (%) | cells (%) |
| 1 | 91 | 84.3 | 0.8 |
| 2 | 94.7 | 78.2 | 4.5 |
| 3 | 98.3 | 89.3 | 2.1 |
| 4 | 96.9 | 80.2 | 5.8 |
| 5 | 96.8 | 90.7 | 0.5 |
| 6 | 92.8 | 82.3 | 0.7 |
| 7 | 95.6 | 88.6 | 3.2 |

Comparison of the recognition of synthetic DTX sequences by CD4+ enriched PBMC and anti-DTD CD4+ cell lines from the same subject. Previous studies on the epitopes recognized by CD4+ cells of normal subjects for TTX and of myasthenic patients for AChR, using unselected PBMC or CD4+ enriched PBMC, found that the responses of unselected blood T cells were low and inconsistent (O'Sullivan et al., J. Immunol., 147, 2663 (1991); Protti et al., J. Immunol., 144, 1276 (1990); Manfredi et al., J. Clin. Invest., 92, 1055 (1993); Manfredi et al., Neurology, 42, 1092 (1992)). This problem is circumvented by the use of Ag-specific CD4+ lines propagated in vitro by stimulation with the relevant Ag.

Ag-specific lines have the important caveat that, due to biased clonal propagation in vitro, they might not be representative of the frequency of the starting clonal repertoire. Therefore, experiments were carried out comparing the responses to individual peptides of CD4+ enriched PBMC and of anti-DTD CD4+ lines from the same subject.

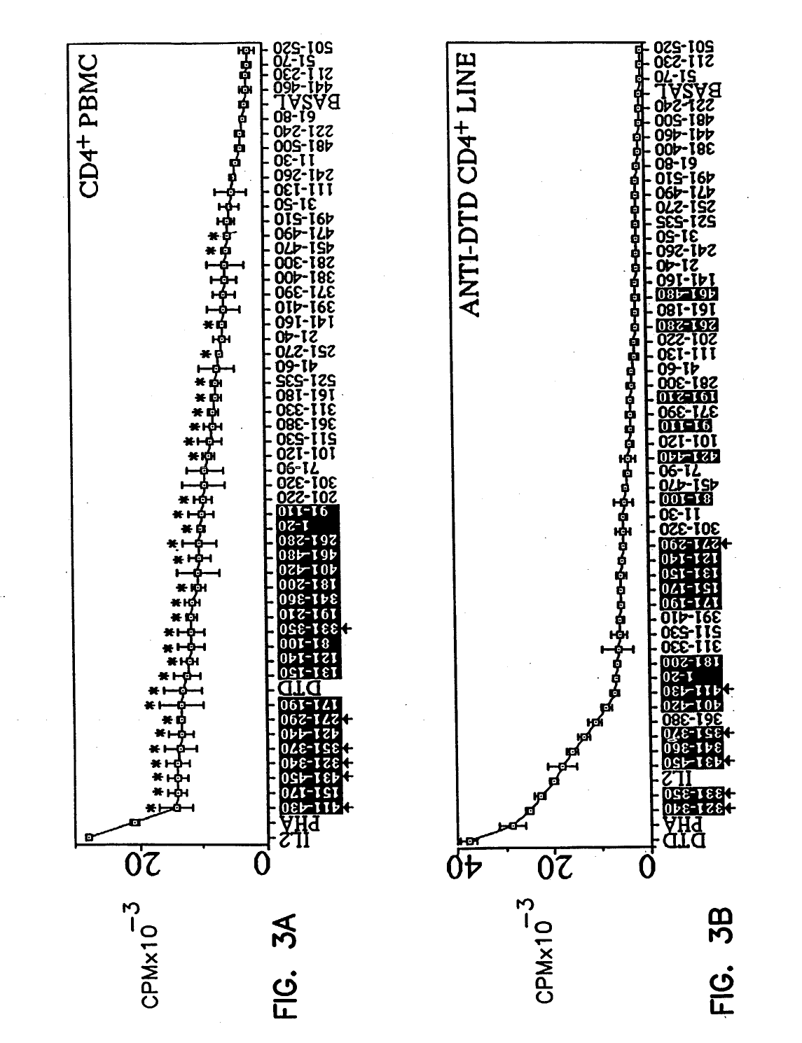

The anti-DTX CD4+ lines propagated from subjects #4 and #5, and their CD4+ enriched PBMC, were tested in proliferation assays with individual peptides. FIG. 3 illustrates the results obtained in one such comparative experiment, using subject #4. The ³H-thymidine incorporation obtained in response to DTD and to DTX-specific peptides was much higher for the CD4+ cell line then for CD4+ enriched PBMC, but the peptides eliciting a positive response were in general the same. Fourteen of the 20 peptides most strongly recognized by CD4+ enriched PBMC of subject #4 were among the 20 peptides most strongly recognized by the CD4+ line from the same subject (see boxed peptide designations in FIG. 3). Four of the remaining six peptides recognized by the CD4+ enriched PBMC were also recognized by the CD4+ line, although to a lesser extent than the peptides described above. Two peptides (421-440 and 461-480) recognized by the CD4+ enriched PBMC of subject #4 were not recognized by the CD4+ line in this experiment, possibly due to replicate scattering, but they were recognized in a second experiment, carried out two weeks later. Similar experiments carried out with subject #5 yielded comparable results.

Therefore, the spectrum of peptides recognized by the CD4+ enriched PBMC and CD4+ lines from the same subjects were qualitatively very similar, but the signal to noise ratio was much better for the CD4+ lines. Therefore, CD4+ lines were used for the following studies.

Sequence segments of DTX recognized by anti-DTD T cell lines. The DTX epitopes recognized by the CD4+ cell lines were identified in proliferation assays using individual synthetic DTX-specific peptides. To minimize the potential loss of epitope recognition resulting from biased clonal propagation, the lines were tested for reactivity to individual synthetic peptides as soon as a satisfactory enrichment in reactivity to DTD was obtained, i.e., when the response of the lines to DTD in a proliferation assay was comparable to that of PHA, usually after 3-4 weeks of culture. The consistency of the recognition was verified by repeating the test 1-2 weeks later. For all lines, very similar or identical patterns of peptide recognition were observed in both experiments.

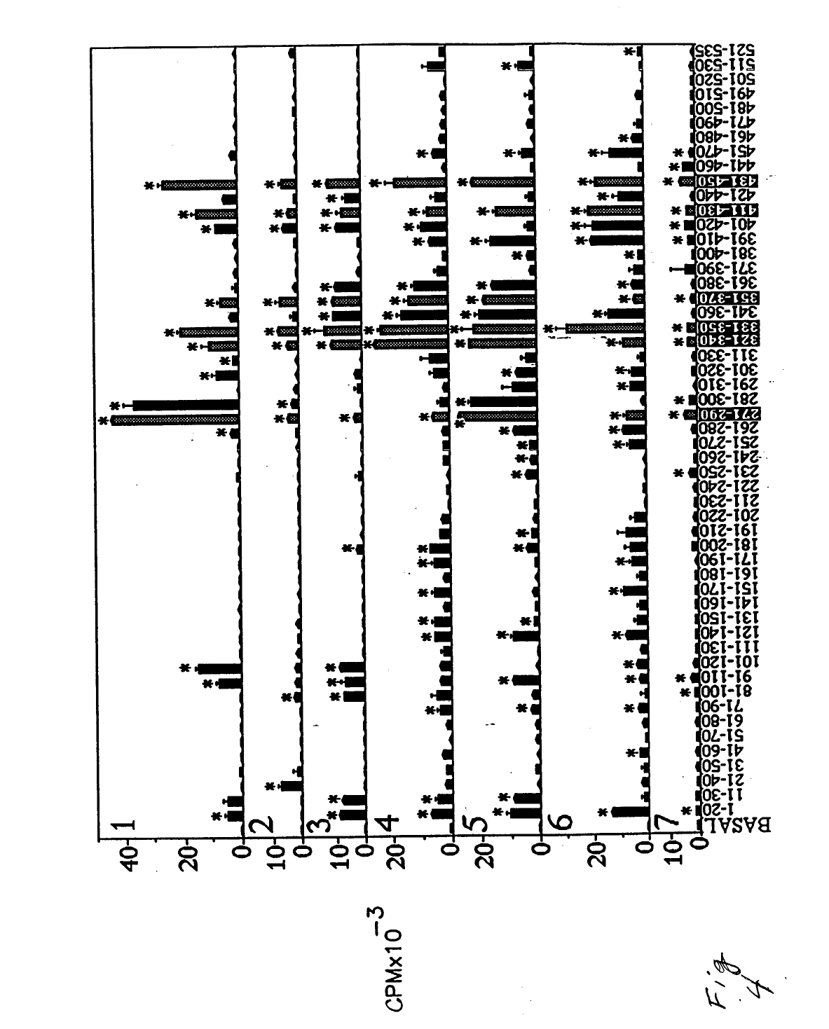

The results obtained in one experiment for each line, testing the response to each individual peptide, are shown in FIG. 4. Although the lines from different subjects showed different degrees of responsiveness to DTX sequences, all subjects recognized a large number of DTX-specific peptide sequences. For all subjects, most of the recognized sequences were within residues 271-450, which form the B fragment, while peptides corresponding to fragment A (amino acids 1-190) were, in general, less immunogenic. Relatively immunodominant sequence segments within fragment A were located at the N terminal region of fragment A, i.e., residues 1-30, and, to a lesser extent, residues 81-120.

Six peptides in the B region were recognized by all seven individuals studied; these peptides comprise residues 271-290 (SEQ ID NO: 2), 321-340 (SEQ. ID NO: 3), 331-350 (SEQ ID NO: 4), 351-370 (SEQ ID NO: 5), 411-430 (SEQ ID NO: 6) and 431-450 (SEQ ID NO: 7). Peptides with residues 321-340 and 331-350 might contain a single CD4+ epitope within their overlap region.

HLA class II restriction. The class II restriction of each IRS was studied in six of the seven subjects, by determining if mAbs against the different class II isotypes affected the response of epitope-specific CD4+ lines to the relevant peptides. As a control for mAb toxicity, the effect of the mAb on the proliferative response induced by IL-2 was determined. As a negative control, triplicate cultures were cultured in the presence of a 20-residue synthetic sequence unrelated to the sequence of peptides with an IRS (residues 1-20 of the TTX light chain sequence).

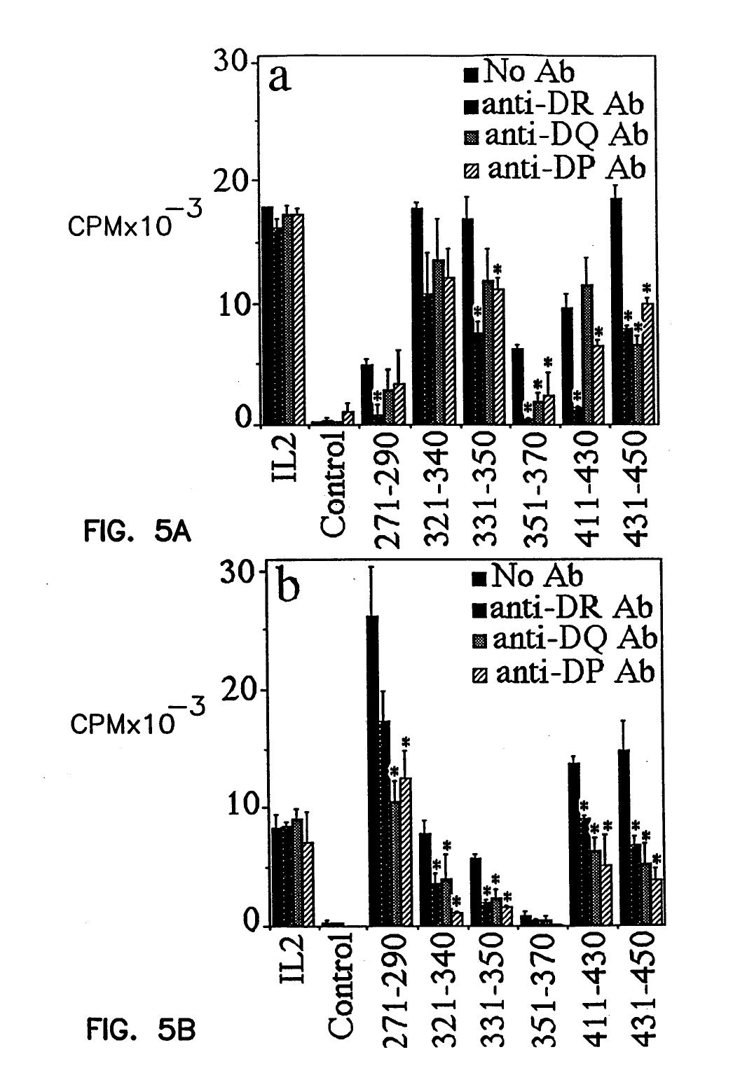

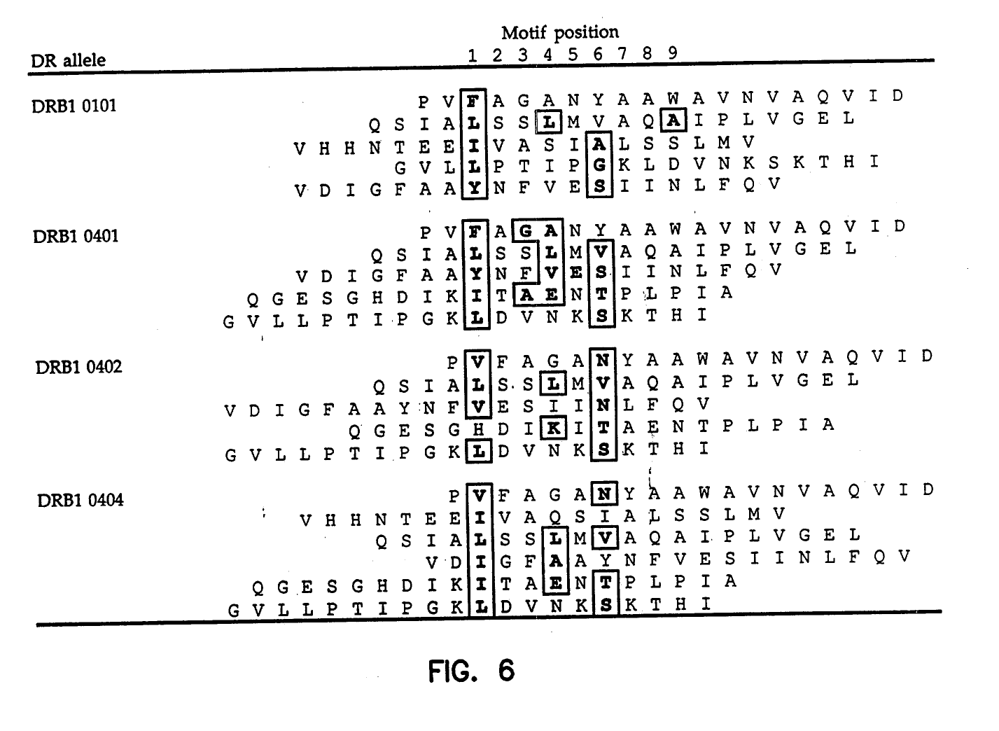

The results obtained in experiments testing the response of the anti-DTD CD4+ cell lines from two subjects, which are representative of those obtained for all subjects, are shown in FIG. 5. As judged by a significant reduction in cell proliferation in the presence of an mAb against a given class II isotype, most IRSs were presented by two, or all three, class II isotypes. A few IRSs, which were different in different subjects, were presented by one isotype only.

Discussion. Thus, a number of DTX sequence regions are recognized by human CD4+ cells in different subjects. Most CD4+ epitopes are clustered in fragment B of DTX. Although each person had a characteristic pattern of peptide recognition (FIG. 4), six DTX peptide sequences, peptides with residues 271-290 (SEQ. ID NO: 2), 321-340 (SEQ. ID NO: 3), 331-350 (SEQ. ID NO: 4), 351-370 (SEQ. ID NO: 5), 411-430 (SEQ. ID NO: 6), and 431-450 (SEQ. ID NO: 7), were recognized by every individual, irrespective of the class II haplotype. Recognition of an IRS accounted for a substantial fraction of the total response of the CD4+ line to DTX sequences (28-57%, see Table 3). In the two subjects where the CD4+ enriched PBMC response to DTX sequences was investigated, all the IRSs were strongly recognized, at levels comparable to the response induced by DTD, in spite of the overall low level of the response of CD4+ enriched PBMC (FIG. 3). The IRS containing peptides were frequently presented by more than one class II isotype (FIG. 5).

TABLE 3

| Subject | % of the total response |
|---|---|
| # | due to the IRS^{a} |
| 1 | 57 |
| 2 | 51 |
| 3 | 42 |
| 4 | 37 |
| 5 | 43 |
| 6 | 31 |
| 7 | 28 |
| ^{a}The fraction of the total response to DTX peptide sequences due to the response to IRS was determined as follows. Each anti-DTD line was challenged with each individual peptide in triplicate cultures. The basal level of cell proliferation, in the absence of any stimulus, was determined in triplicate cultures containing T line cells and APC only (blanks). The average cpm values obtained for the blanks were | |
| # subtracted from those obtained for the peptides. The values thus obtained were added up, to yield what was considered 100% stimulation by DTX-specific peptides. The average cpm obtained for each IRS minus blank were added, and the fraction of the total response which this sum represented was calculated. | |

Preferential recognition of certain epitopes might be due to a biased V (variable) region repertoire of the TCR expressed by that subject, or by all subjects expressing a given class II haplotype. However, this explanation does not hold for the findings reported here, because the IRSs were recognized by subjects of different MHC class II haplotype. The molecular basis of the preferred recognition of the IRS by human CD4+ cells could be due to the characteristics of the interaction of peptide epitopes with HLA class II molecule, and/or the structural properties of the Ag molecule, which may influence processing and presentation of certain sequence regions.

Many peptide sequences can bind different DR alleles. Nonetheless, the ability of a given Ag sequence to bind most or all DR molecules does not suffice for a peptide to be an IRS (for example, see Manfredi et al., J. Immunol., 152, 4165 (1994); Reece et al., J. Immunol., 151, 6175 (1993)). Factor(s) that are important for an Ag sequence region to be an IRS for CD4+ cell sensitization include a structural property which gives the IRS an advantage during Ag processing, causing its preferential release from the Ag molecule, and/or availability for class II binding and presentation.

DTX has three distinct domains (Choe et al., supra): the C or catalytic domain (residues 1-193), which is formed by fragment A, the T or transmembrane domain (residues 205-378), and the R or receptor binding domain (residues 386-535). The T and the R domains form fragment B. All IRSs described above are within fragment B: residues 411-430 and 431-450 are part of the R domain, the others are part of the T domain. The T domain includes nine α helices (TH1-TH9), arranged in three antiparallel layers. Helices TH8 and TH9 are unusually apolar and constitute the central core layer. The R domain consists of nine β strands (RB1-RB9). These secondary structure elements are connected by loops. All IRSs include one or more of the a helixes or β sheets described above.

A common structural property of the IRSs that may give them an advantage during DTX processing is that they all include, or are flanked by, both at the amino and carboxyl terminal ends, sequence regions forming relatively unstructured loops fully exposed to the solvent. These loops may be easily accessible targets for the proteolytic enzymes involved in Ag processing, even in the absence of any substantial denaturation of the Ag.

For example, IRS peptide 271-290 (SEQ ID NO: 2) includes the α helix TH5 (residues 275-288), one face of which is exposed to the solvent. TH5 is flanked on its amino terminal end by an exposed loop formed by residues 271-274, and at its carboxyl terminal end by another exposed loop, formed by residues 289-296.

The overlapping IRS peptides 321-340 (SEQ. ID NO: 3) and 331-350 (SEQ ID NO: 4) (sequence 321-350) include the helix TH8 (residues 326-347), which, although contained in the core of the native DTX molecule, is flanked at both its amino and carboxyl terminal ends by solvent-exposed loops, formed by residues 322-327 and 348-357, respectively. These two overlapping IRs peptides might include only one epitope, within the sequence region forming TH8, which includes the overlap between peptides 321-340 and 331-350 (residues 331-340). In the native DTX molecule, this epitope is flanked by fully exposed loops at either end.

IRS peptide 351-370 (SEQ ID NO: 5) includes the majority of the α helix TH9 (residues 358-376), which, although mostly buried in the core of the DTX molecule (it has only one exposed residue), is flanked at both ends by fully exposed loops, namely, the coil regions formed by residue 348-357, between helices TH8 and TH9, and 377-388.

IRS peptide 411-430 (SEQ ID NO: 6) includes the β strand RB3 (residues 413-422), several residues of which (423-426) are fully exposed to the solvent. RB3 is preceded, on its amino terminal side, by an exposed loop formed by residues 408-412. Also, at the carboxyl terminal end of IRS 411-430, residues 423-431 form an exposed loop connecting RB3 to RB4.

IRS peptide 431-450 (SEQ ID NO: 7) includes the β strand RB4 (431-443). RB4 is followed by an exposed loop (residues 444-448) and is preceded in the DTX molecule by a small exposed loop between RB3 and RB4 (423-431).

Therefore, the present results show that sequence segments “hidden” in the hydrophobic core of a protein Ag might also be important targets of immune recognition by CD4+ cells because helices TH8 and TH9, which correspond to IRS 321-340, 331-350, and 351-370, are deep in the core of the DTX molecule. This underscores the importance of flanking exposed loops for IRS formation. These exposed loops would make an easy target for processing enzymes, resulting in the fast release of sequence segments embedded in the hydrophobic core of the Ag molecule.

Because of the IRSs are recognized in association with different class II alleles and isotypes, their sequence must have characteristics compatible with binding to a large number of different class II molecules. X-ray diffraction studies of the DR1 molecule indicated that several residues involved in formation of the peptide binding site are conserved in most or all class II isotypes, suggesting that all class II molecules bind peptides with similar mechanism (Stern et al., Nature, 368, 215 (1994); Brown et al., Nature, 364, 33 (1993). In agreement with that prediction, the DTX IRSs were frequently recognized in association with different class II isotypes.

Previous studies, based on sequence alignments of naturally processed peptides, eluted from purified DR molecules, or on the effect on binding to DR of substitutions of individual residues within a peptide sequence, suggested sequence motifs that could be characteristic of binding to a given DR allele, or of “universal” DR binding. Crystallographic studies of the DR1 molecule complexed to a peptide, and binding studies utilizing phage display libraries, thus directly studying any possible sequence of a given length, have identified the structural and sequence properties necessary for a peptide to bind to different DR types (Stern et al., supra; Hammer et al., Cell, 74, 197 (1993); Wicherpfenning et al., J. Exp. Med., 181, 1597 (1995); Geluk et al., Eur. J. Immunol., 22, 107 (1995); Hammer et al., J. Exp. Med., 181, 1847 (1995).

Peptides bind to DR molecules in an extended conformation, which allows extensive hydrophobic interactions between the peptide backbone and the binding groove of the DR molecules, thus providing a mode of peptide binding independent of the peptide sequence (Stern et al., supra; Jardetzky et al., EMBO, 9, 1797 (1990)). Peptide specificity is due to interactions between pockets on the DR molecules, whose surface have a shape and charges characteristic for a given DR allele, and to anchor residues of suitable size, hydropathic properties and charge (Stern et al., supra; Hammer et al., supra).

Although as many as seven anchor residues have been identified, at least for a DR4 subtype, only one or very few residues are crucial for binding (Hammer et al., PNAS USA, 91, 4456 (1994)), and the others, while improving the affinity of the binding, tolerate a broad range of substitutions, without obliterating the peptide/DR interaction (Hammer et al., J. Exp. Med., supra; Hammer et al., PNAS USA, supra). While anchor residues are frequently uncharged or hydrophobic, both positively and negatively charged anchor residues have been identified for peptide binding to individual DR alleles, fitting in pockets, on the DR molecule, lined by residues of complementary charge. When the lining of DR binding pockets may have charges, the presence of the wrong charge on a peptide residue aligned with that pocket may de-stabilize peptide-DR binding.

While it is unknown which residues within the IRS interact with the different class II molecules, and structural correlates between the sequence of an IRS peptide and its ability to bind to different presenting molecules are not identified, the binding motifs identified for peptide binding to DR1 (Hammer et al., J. Exp. Med., supra), and different DR4 subtypes (Hammer et al., Cell, supra; Hammer et al., J. Exp. Med., supra; Jardetzky et al., supra; Sette et al., J. Immunol., 151, 3163 (1993)) present in most or all the DTX IRSs are shown in Table 4.

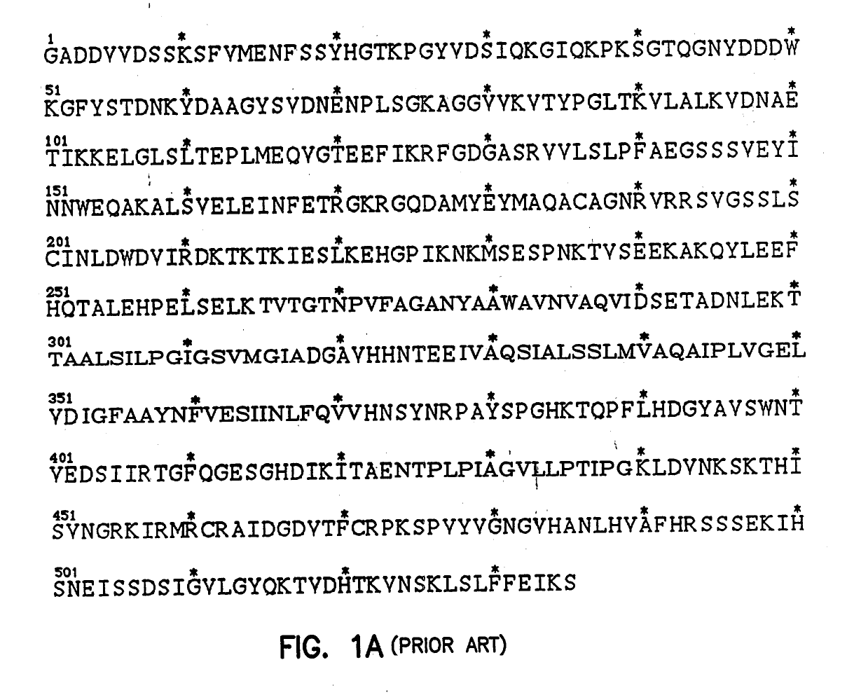

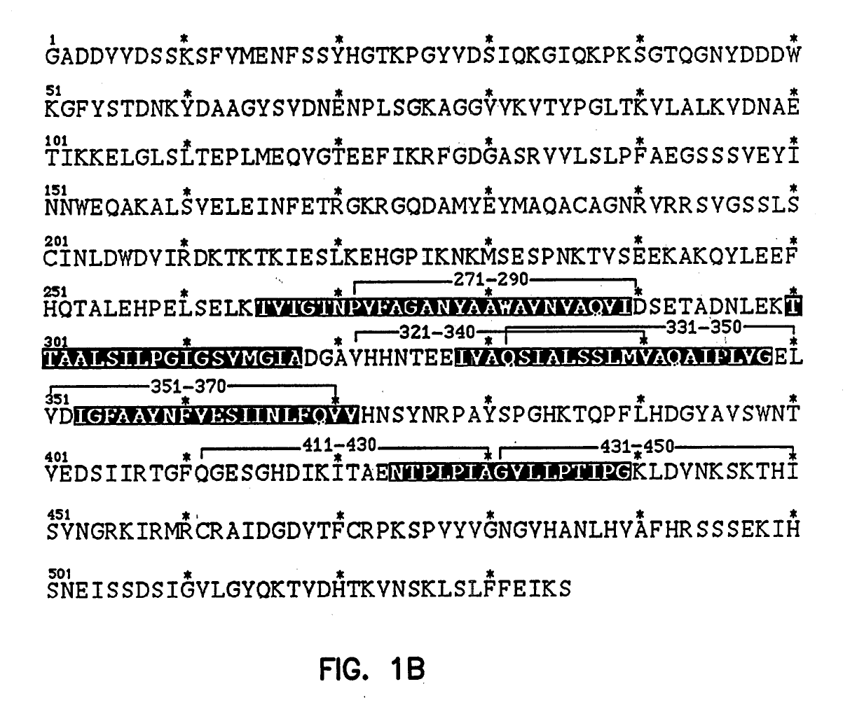

All the IRSs identified here overlap four of the five DTX sequence segments which are most hydrophobic: four of those segments do not contain any charged residue, and one (segment 353-371) contains a single charge (London et al., Biochem. Biophys. Acta., 1113, 25 (1992)). The relationship between the IRSs and those uncharged DTX sequence regions is illustrated in FIG. 1. However, the uncharged nature of a DTX peptide sequence is not predictive of an IRS, because some peptides which largely overlapped an uncharged sequence region were not recognized by all the subjects. Also, all the IRSs included residues outside the hydrophobic regions described above, some of which are charged.

However, it is possible that the presence of a stretch of uncharged residues might be related with IRS formation, as uncharged sequence segments might be preferred as “universal” DR binders because the uncharged residues would not have any negative effect on binding. This is in contrast to charged residues which would carry a “wrong” charge that would strongly, negatively effect peptide binding to some class II molecules.

### EXAMPLE 2

PBMC of 49 randomly selected, HLA heterogenous human subjects were challenged with each of the identified DTX and TTX universal epitope peptides. The results are summarized below in Table 4. Generally, the PBMCs of each subject recognized several universal epitopes and all but one recognized at least one of the peptides. Although most of the epitopes are recognized in this assay, the lack of recognition by PBMC of a particular peptide does not exclude that the T cells recognize the peptide, as this assay has low sensitivity.

| | | | | | | | | | TTD sequence | | |
|---|---|---|---|---|---|---|---|---|---|---|---|
| | | DTD sequence segment | | | | | | | segment | | |
| Control | HLA | | 271- | 321- | 331- | 351- | 411- | 431- | | 176- | 491- |
| # | (DR) | DTD | 290 | 340 | 350 | 370 | 430 | 450 | TDD | 195 | 510 |
| 1 | 6, 8 | + | + | + | + | + | + | − | + | + | + |
| 2 | 2, 3 | + | + | + | + | + | + | + | + | + | + |
| 3 | 3, 3 | + | + | − | − | + | − | + | + | + | + |
| 4 | 4, 6 | + | + | + | + | + | + | + | + | + | + |
| 5 | 3, 7 | + | + | + | + | + | + | + | + | + | + |
| 6 | 2, 3 | + | + | + | − | + | + | + | + | + | + |
| 7 | 6, 6 | + | + | − | − | + | + | + | + | + | + |
| 8 | 2, 4 | + | + | + | + | + | + | + | + | + | + |
| 9 | 1, 4 | + | + | + | + | + | + | + | + | + | + |
| 10 | 1, 4 | + | + | − | − | − | − | − | + | − | − |
| 11 | 2, 4 | + | + | + | + | + | + | + | + | + | + |
| 12 | 4, w13(6) | + | + | + | + | + | + | + | + | + | + |
| 13 | 1, w12(5) | + | + | + | + | − | + | + | + | + | + |
| 14 | 2, 2 | + | + | + | − | − | − | − | − | + | − |
| 15 | w15(2), | − | + | + | + | − | + | + | + | + | + |
| | w17(3) | | | | | | | | | | |
| 16 | 4, w12(5) | + | + | + | + | + | + | + | + | + | + |
| 17 | 4, w11(5) | + | + | + | + | − | + | + | + | + | + |
| 18 | w15(2), 4 | + | + | + | + | + | + | + | + | + | + |
| 19 | 4, 4 | + | + | + | + | − | + | − | + | + | + |
| 20 | 5, 7 | + | + | + | + | − | + | + | + | + | + |
| 21 | w17(3), 7 | + | + | + | + | + | + | + | + | + | + |
| 22 | 3, 7 | + | + | − | + | + | + | − | + | + | + |
| 23 | 4, 8 | + | + | + | + | + | − | − | + | + | − |
| 24 | 1, 6 | + | + | − | − | − | − | − | + | − | − |
| 25 | 4, 4 | + | + | − | − | − | − | − | + | + | + |
| 26 | 4, 5 | − | + | − | + | + | − | + | + | + | + |
| 27 | 3, 5 | + | + | + | + | + | + | + | + | + | + |
| 28 | 2, 10 | + | + | − | + | − | − | − | + | − | − |
| 29 | 3, 5 | + | + | − | − | − | − | − | + | + | + |
| 30 | 6, 7 | + | − | + | + | + | + | + | + | + | + |
| 31 | 6, 7 | − | + | + | + | + | − | + | + | + | + |
| 32 | 6, 7 | + | + | + | + | + | + | − | + | + | + |
| 33 | 1, w15(2) | + | + | + | + | + | + | + | + | + | + |
| 34 | w15(2), | + | + | + | − | + | − | − | + | + | + |
| | w17(3) | | | | | | | | | | |
| 35 | 3, 6 | + | + | + | + | + | + | + | + | + | + |
| 36 | 2, 2 | + | + | + | + | + | + | + | + | + | + |
| 37 | 4, 7 | + | + | + | + | + | + | + | + | + | + |
| 38 | 3, 8 | + | − | + | + | − | − | + | + | + | − |
| 39 | 3, 7 | + | + | − | − | + | + | + | − | + | + |
| 40 | 3, w14(6) | + | − | + | + | + | + | + | + | + | + |
| 41 | 4, 5 | + | + | − | + | + | + | − | + | − | + |
| 42 | 1, 4 | + | + | + | + | na | + | + | + | − | + |
| 43 | 1, 7 | + | − | + | + | na | − | + | + | + | + |
| 44 | 4, w11(5) | + | + | + | + | na | + | − | + | + | + |
| 45 | 5, 5 | + | − | − | + | na | − | + | + | + | + |
| 46 | w13(6), | + | + | + | + | na | + | + | + | + | − |
| | w15(2) | | | | | | | | | | |
| 47 | 1, 7 | + | + | + | + | na | + | + | + | + | + |
| 48 | 4, 6 | + | − | + | + | na | − | + | + | + | + |
| 49 | 3, 3 | + | − | + | + | na | + | − | + | + | + |
| positive | | 95 | 84 | 76 | 80 | 71 | 70 | 70 | 96 | 90 | 86 |
| response | | | | | | | | | | | |
| (1%) | | | | | | | | | | | |

### EXAMPLE 3

Five T cell clones from the same subject were challenged with DTX peptides. Three of the clones were obtained after stimulation with DTX universal epitope peptide 271-290, and two of the clones were obtained after stimulation with DTX universal epitope peptide 411-430. The clones reacted with all the universal DTX epitope peptides tested (FIG. 9). Although the clones were obtained from polyclonal lines specific for DTX peptide 271-290 and DTX 411-430 and those polyclonal lines recognized DTD vigorously, the clones did not effectively recognize DTD. Possible explanations for this observation include a low affinity binding of DTD by the clones which was insufficient for clonal stimulation of the amount of epitope processed from the small amount of DTD in the culture, insufficient presentation of DTD by antigen presenting cells, or that the use of a peptide to propagate the cultures leads to clones that recognize cryptic epitopes.

The Vβ region of each of the three clones to peptide 271-290 were determined. Two of the clones used Vβ7 and the other clone used Vβ7 and Vβ17. The usage of two Vβ families may be due to the presence of two clones in the clonal population or due to allelic exclusion.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. An isolated and purified peptide comprising an amino acid sequence that is substantially similar or identical to a portion of the amino acid sequence of an antigen from an infectious agent, wherein the antigen is present on the surface of the agent, wherein the infectious agent is a virus, bacterium or fungus, wherein the peptide is between about 7 and about 40 amino acid residues in length, and wherein the peptide comprises a universal epitope sequence.

2. The peptide of claim 1 which comprises an immunodominant region sequence.

3. A method to identify an immunogenic epitope, comprising:
(a) exposing cultured immune cells to at least one isolated and purified peptide, wherein the amino acid sequence of the peptide is substantially similar or identical to a portion of the amino acid sequence of an antigen of an infectious agent, wherein the antigen is present on the surface of the agent; and
(b) determining whether or not the cultured immune cells proliferate relative to control immune cells which were not exposed to the peptide or any other antigenic stimulus.

4. The method of claim 3 wherein the infectious agent is a virus, bacterium or fungus.

5. The method of claim 3 wherein the immune cells are mammalian immune cells.

6. The method of claim 5 wherein the immune cells are peripheral blood mononuclear cells, spleen cells or lymph node cells.

7. The method of claim 5 wherein the peptide is synthesized in vitro.

8. The method of claim 5 wherein the immune cells were previously stimulated in vitro.

9. The method of claim 5 wherein the immune cells were previously stimulated in vivo.

10. The method of claim 5 wherein the cultured immune cells are depleted of CD8+ cells.

11. The method of claim 5 wherein the cultured immune cells were previously stimulated with the infectious agent, the antigen, or antigen-specific peptides to yield a CD4+ T cell line which is specific for antigen-specific epitopes or enriched in CD4+ cells specific for antigen-specific epitopes.

12. The method of claim 5 wherein the peptide comprises at least 7 amino acid residues.

13. A method to identify an immunodominant region sequence in a peptide, comprising:
(a) exposing each of at least a first and a second culture of immune cells to at least one isolated and purified peptide, wherein the HLA or MHC haplotype of the immune cells in at least the first and second of the cultures is different, and wherein the amino acid sequence of the peptide is substantially similar or identical to a portion of the amino acid sequence of an antigen of an infectious agent, wherein the antigen is present on the surface of the agent; and
(b) determining whether or not the immune cells in any of the exposed cultures proliferates relative to control immune cells which were not exposed to the peptide or any other antigenic stimulus.

14. The method of claim 13 wherein the peptide is synthesized in vitro.

15. The method of claim 13 wherein the immune cells were previously stimulated in vitro.

16. The method of claim 13 wherein the immune cells were previously stimulated in vivo.

17. The method of claim 13 wherein the cultured immune cells were previously stimulated with the infectious agent, the antigen, or antigen-specific peptides to yield CD4+ T cells which are specific for antigen-specific epitopes.

18. The method of claim 13 wherein the peptide comprises at least 7 amino acid residues.

19. The method of claim 13 wherein the immune cells are depleted of CD8+ cells.

20. A vaccine comprising an immunogenic amount of at least one peptide containing a universal epitope sequence, wherein the peptide comprises an amino acid sequence substantially similar or identical to a portion of the amino acid sequence of an antigen from an infectious agent, wherein the antigen is present on the surface of the agent, wherein the peptide is combined with a physiologically acceptable, non-toxic liquid vehicle, which amount is effective to immunize a susceptible mammal against the infectious agent.

21. The vaccine of claim 20 wherein the mammal is a human.

22. The vaccine of claim 20 which further comprises a carrier.

23. The vaccine of claim 22 wherein the carrier comprises an amount of the antigen.

24. An immunogenic composition comprising a peptide associated with a non- or poorly immunogenic molecule, wherein the peptide comprises an amino acid sequence substantially similar or identical to a portion of the amino acid sequence of an antigen from an infectious agent, wherein the antigen is present on the surface of the agent, wherein the peptide is between 7 and 40 amino acid residues in length, and wherein the peptide comprises an immunodominant or universal epitope sequence.

25. An immunogenic composition comprising a peptide associated with a non- or poorly immunogenic molecule, wherein the peptide consists essentially of an amino acid sequence region that is present on the surface of crystallized surface antigen of an infectious agent, and wherein the peptide comprises an immunodominant or universal epitope sequence.

26. A method to identify an immunogenic epitope, comprising:
(a) exposing cultured immune cells to at least one isolated and purified peptide, wherein the amino acid sequence of the peptide is substantially similar or identical to a portion of the amino acid sequence of an antigen that is present on the surface of an infectious agent; and
(b) determining whether or not the cultured immune cells produce at least one cytokine relative to control immune cells which were not exposed to the peptide or any other antigenic stimulus.

27. The method of claim 26 wherein the peptide is synthesized in vitro.

28. The method of claim 26 wherein the immune cells were previously stimulated in vitro.

29. The method of claim 26 wherein the immune cells were previously stimulated in vivo.

30. The method of claim 26 wherein the cultured immune cells are depleted of CD8+ cells.

31. The method of claim 26 wherein the cultured immune cells were previously stimulated with the infectious agent, the antigen, or antigen-specific peptides to yield a CD4+ T cell line which is specific for antigen epitopes or enriched in CD4⁺ cells specific for antigen epitopes.

32. The method of claim 26 wherein the peptide comprises at least 7 amino acid residues.

33. The method of claim 26 wherein the cytokine is IL-2, IL-3, IL-4, IL-5, IL-10 or gamma interferon.

34. A method to identify an immunodominant region sequence in a peptide, comprising:
(a) exposing each of at least a first and a second culture of immune cells to at least one isolated and purified peptide, wherein the HLA or MHC haplotype of the immune cells in at least the first and second of the cultures is different, and wherein the amino acid sequence of the peptide is substantially similar or identical to a portion of the amino acid sequence of an antigen present on the surface of an infectious agent; and
(b) determining whether or not the immune cells in any of the exposed cultures produce at least one cytokine relative to control immune cells which were not exposed to the peptide or any other antigenic stimulus.

35. The method of claim 34 wherein the peptide is synthesized in vitro.

36. The method of claim 34 wherein the immune cells were previously stimulated in vitro.

37. The method of claim 34 wherein the immune cells were previously stimulated in vivo.

38. The method of claim 34 wherein the cultured immune cells were previously stimulated with the infectious agent, the antigen, or antigen-specific peptides to yield CD4+ T cells which are specific for antigen epitopes.

39. The method of claim 34 wherein the peptide comprises at least 7 amino acid residues.

40. The method of claim 34 wherein the immune cells are depleted of CD8+ cells.

41. The method of claim 34 wherein the cytokine is IL-2, IL-3, IL-4, IL-5, IL-10 or gamma interferon.

42.

43. A method to identify an immunodominant antigen of an infectious agent, comprising:
a) contacting a mammal with an amount of the infectious agent;
b) obtaining serum from the mammal of step (a) and determining which antigen of the infectious agent binds to antibodies present in the serum;
c) purifying antigens that are strongly recognized by the antibodies; and
d) contacting isolated T cells from the mammal of step (a) with an amount of the purified antigen of step (c) and identifying whether the T cells proliferate in response to the purified antigen.

44. A method to identify a universal epitope sequence in a peptide, comprising:
(a) exposing each of at least a first and a second culture of immune cells to at least one isolated and purified peptide, wherein the HLA or MHC haplotype of the immune cells in at least the first and second of the cultures is different, and wherein the amino acid sequence of the peptide is substantially similar or identical to a portion of the amino acid sequence of an antigen of an infectious agent, wherein the antigen is present on the surface of the agent; and
(b) determining whether or not the immune cells in any of the exposed cultures proliferates relative to control immune cells which were not exposed to the peptide or any other antigenic stimulus.

45. The method of claim 44 wherein the peptide is synthesized in vitro.

46. The method of claim 44 wherein the immune were previously stimulated in vitro.

47. The method of claim 44 wherein the immune cells were previously stimulated in vivo.

48. The method of claim 44 wherein the cultured immune cells were previously stimulated with the infectious agent, the antigen, or antigen-specific peptides to yield CD4+ T cells which are specific for antigen-specific epitopes.

49. The method of claim 44 wherein the peptide comprises at least 7 amino acid residues.

50. The method of claim 44 wherein the immune cells are depleted of CD8+ cells.

51. A method to identify a universal epitope sequence in a peptide, comprising:
(a) exposing each of at least a first and a second culture of immune cells to at least one isolated and purified peptide, wherein the HLA or MHC haplotype of the immune cells in at least the first and second of the cultures is different, and wherein the amino acid sequence of the peptide is substantially similar or identical to a portion of the amino acid sequence of an antigen of an infectious agent, wherein the antigen is present on the surface of the agent; and
(b) determining whether or not the immune cells in any of the exposed cultures produce at least one cytokine relative to control immune cells which were not exposed to the peptide or any other antigenic stimulus, determining whether or not the immune cells in any of the exposed cultures proliferates relative to control immune cells which were not exposed to the peptide or any other antigenic stimulus.

52. The method of claim 51 wherein the peptide is synthesized in vitro.

53. The method of claim 51 wherein the immune were previously stimulated in vitro.

54. The method of claim 51 wherein the immune cells were previously stimulated in vivo.

55. The method of claim 51 wherein the cultured immune cells were previously stimulated with the infectious agent, the antigen, or antigen-specific peptides to yield CD4+ T cells which are specific for antigen-specific epitopes.

56. The method of claim 51 wherein the peptide comprises at least 7 amino acid residues.

57. The method of claim 51 wherein the immune cells are depleted of CD8+ cells.

58. The method of claim 13, 34, 44 or 51 wherein the peptide comprises an amino acid sequence of an antigen selected from the group consisting of hemagglutinin (HA) of influenza, the G or F protein of Respiratory Syncytial Virus (RSV), herpes glycoprotein D, surface glycoprotein of rabies virus, the glycoprotein of a retrovirus, and lentiviruses such as HIV, and antigens of Vibrio cholerae and BCG.

59. The method of claim 13, 34, 44 or 51 wherein the peptide comprises an amino acid sequence of an antigen of Vaccinia, smallpox virus, Varicella zoster virus, Polio virus, Cytomegalovirus, Hepatitis A virus, Adenovirus, Influenza virus, Yellow fever virus, Mumps virus, Dengue virus, Hepatitis B virus, Japanese B encephalitis virus, Rabies virus, Rotavirus, Herpes simplex viruses 1 and 2, Herpesvirus varicellae, and Parainfluenza virus, Mycobacterium leprae, Vibrio cholerae, Salmonella typhi, Bordetella pertussis, Streptococcus pneumoniae (pneumococcus), Hemophilus influenzae (type B), Clostridium tentani, Corynebacterium diphtheriae, Coccidioides immitis, Neisseria gonorrhoeae,Streptococcus group B, Plasmodium spp., Escherichia coli, Shigella spp., Streptococcus group A, and Neisseria meningitidis.

60. The method of claim 13, 34, 44 or 51 wherein the peptide comprises an amino acid sequence of an antigen of Pneumococcus, Rotavirus, group A Streptococcus, hepatitis C, poliovirus, Clostridium tentani, Corynebacterium diphtheriae, Mycobacterium tuberculosis, hantavirus, Ebola virus and other viruses causing hemorrhagic fever, Pertussis, Rubella, hepatitis A, and hepatitis B.

61. The method of claim 13, 34, 44 or 51 wherein the peptide comprises an amino acid sequence of liver-specific antigen-1 (LSA-1), thrombospondin-related anonymous protein (TRAP), gp190, Pfs25, Pfs28, Pf155/RESA, GLURP, MSP-1, Pfs48/45, SSP-2, Pfs230, Spf66, or PfEMP1.

62. The method of claim 13, 34, 44 or 51 wherein the peptide comprises an amino acid sequence of an antigen of Plasmodium vivax, Plasmodium ovale or Plasmodium malariae.

63. The method of claim 13, 34, 44 or 51 wherein the peptide comprises an amino acid sequence of a Schistosoma antigen.

64. The method of claim 13, 34, 44 or 51 wherein the peptide comprises an amino acid sequence of an antigen of S. japonicum, S. mansoni or S. hematobulin.

65. The method of claim 13, 34, 44 or 51 wherein the antigen is a Mycobacterium antigen.

66. The method of claim 13, 34, 44 or 51 wherein the peptide comprises an amino acid sequence of an antigen from Lassa virus, yellow fever virus, dengue virus, Junin virus, Machupo virus, LCM virus, hantavirus, Marburg virus or Ebola virus.

67. The method of claim 13, 34, 44 or 51 wherein the peptide comprises an amino acid sequence of an antigen from an Arenavirus.
